(19) United States
(12) Patent Application Publication
(10) Pub. No.: US 2012/0100079 A1
(43) Pub. Date: Apr. 26, 2012

(54) STIMULI-RESPONSIVE CARRIERS FOR MPI-GUIDED DRUG DELIVERY

(75) Inventors: Dirk Burdinski, Essen (DE); Jeroen A. Pikkemaat, Waalre (NL); Bertrand Schmitt, Riedisheim (FR); Holger Gruell, Eindhoven (NL); Sander Langereis, Mierlo (NL)

(73) Assignee: KONINKLIJKE PHILIPS ELECTRONICS N.V., EINDHOVEN (NL)

(21) Appl. No.: 13/379,056

(22) PCT Filed: Jun. 25, 2010
(86) PCT No.: PCT/IB10/52918

§ 371 Date: Dec. 19, 2011

(30) Foreign Application Priority Data
Jul. 1, 2009 (EP) 09164301.5

(51) Int. Cl. A61K 49/18; A61P 41/00

(52) U.S. Cl. 424/9.321; 424/9.32

(57) ABSTRACT

The present invention relates to a composition comprising a shell structure forming a cavity, wherein said shell structure comprises a drug and wherein said composition is associated with at least one contrast agent; wherein said shell structure is capable of releasing its contents into the exterior upon the application of an external stimulus and wherein said contrast agent comprises magnetic particles which are capable of being detected by Magnetic Particle Imaging (MPI), wherein at least more than 5% (w/w) of the magnetic particles comprised in said contrast agent have a magnetic moment of at least −18 m 2 A, 10 wherein said magnetic particles are preferably composed of Fe, Co, Ni, Zn or Mn or alloys thereof or oxides of any of these. The present invention further relates to the use of such a composition or a composition comprising a shell structure forming a cavity, wherein said shell structure comprises a drug and wherein said composition is associated with at least one contrast agent, wherein said contrast agent is capable of being detected by MPI and wherein said shell structure is capable of releasing its contents into the exterior upon the application of an external stimulus as a carrier for a controlled delivery of a drug, as well as to a method of data acquisition for the control of a drug delivery process comprising the detection or localization via MPI of such compositions. In a further aspect the present invention relates to such compositions for treating a pathological condition, wherein the treatment comprises the release of the drug by the application of a stimulus.

### FIELD OF THE INVENTION

The present invention relates to a composition comprising a shell structure forming a cavity, wherein said shell structure comprises a drug and wherein said composition is associated with at least one contrast agent; wherein said shell structure is capable of releasing its contents into the exterior upon the application of an external stimulus and wherein said contrast agent comprises magnetic particles which are capable of being detected by Magnetic Particle Imaging (MPI), wherein at least more than 5% (w/w) of the magnetic particles comprised in said contrast agent have a magnetic moment of at least 10⁻¹⁸ m²A, wherein said magnetic particles are preferably composed of Fe, Co, Ni, Zn or Mn or alloys thereof or oxides of any of these. The present invention further relates to the use of such a composition or a composition comprising a shell structure forming a cavity, wherein said shell structure comprises a drug and wherein said composition is associated with at least one contrast agent, wherein said contrast agent is capable of being detected by MPI and wherein said shell structure is capable of releasing its contents into the exterior upon the application of an external stimulus as a carrier for a controlled delivery of a drug, as well as to a method of data acquisition for the control of a drug delivery process comprising the detection or localization via MPI of such compositions. In a further aspect the present invention relates to such compositions for treating a pathological condition, wherein the treatment comprises the release of the drug by the application of a stimulus.

### BACKGROUND OF THE INVENTION

Drug delivery is a compilation of different methods or processes for administering a pharmaceutically, therapeutically or diagnostically useful compound to achieve a medical effect in humans or animals. Drug delivery technologies can modify drug release profiles, absorption, distribution and/or elimination of drugs for the benefit of improving product efficacy and safety, as well as patient convenience and compliance. Classical drug delivery can make use of, inter alia, non-invasive peroral, topical, transmucosal and inhalation routes. Typical drug delivery strategies are based on a systemic application of the drug often bringing about significant side effects for the patient due to undesired biodistribution and toxicity. An important drawback of such systemic approaches is the fact that the therapeutic efficiency is dependent on the minimal required drug concentration in the diseased or target tissue or organ on the one hand and the toxic effects of the drug in non-targeted parts of the body on the other.

In order to overcome this difficulty, local and stimulated drug delivery based on carriers, e.g. liposome or polymer micelle carriers, was developed as a new facet in the field of drug delivery. This technique offers significant advantages over classical, systemic disease treatment protocols since the local concentration of the medicament can be increased and, at the same time, adverse systemic side-effects can be avoided. Localized drug delivery may, thus, be the option of choice for many diseases or pathological conditions, in particular if other therapeutic approaches such as surgery are not feasible or too risky.

Carrier-based drug delivery is typically performed by first loading a carrier with a drug or substance of choice and a subsequent release of said substance or drug load upon the application of an external trigger, e.g. a temperature or pressure stimulus, at a desired location (Torchilin, 2005, Nature Reviews Drug Discovery, 4, 145-160).

Techniques for carrier-based drug delivery have successfully been combined with the use of contrast agents. For example, stimuli-responsive liposomes were linked with Magnetic Resonance Imaging (MRI) contrast agents by encapsulating the contrast agent in the lumen of the liposomes (McDannold et al., 2004, Radiology, 230, 743-752). MRI is an important diagnostic technique which is commonly used in hospitals for diagnostic purposes and allows for the non-invasive imaging of soft tissue with a high spatial resolution. The technique is based on the imaging of bulk water molecules, which are present at a very high concentration throughout the body in all tissues. As contrast agents, complexes of gadolinium or manganese ions are used, which reduce the longitudinal (T₁) and transverse (T₂) relaxation times of the protons of water molecules. Due to its capacity, MRI was shown to allow the monitoring of the delivery of substances, e.g. drugs, comprised in carrier structures.

However, in such an approach the initial contrast agent concentration inside the carrier is so high that the concentration of these carriers cannot be determined easily in the beginning of the intervention due to a significant T₂ shortening and diffusion effects. Only upon heating, the T₁ contrast agents are liberated and diluted to provide a positive contrast in MRI. Similar considerations apply to combinations of drug delivery carriers and contrast agents comprising T₂* Magnetic Resonance (MR) contrast agents, or ¹⁹F tracers. These alternative approaches provide either problems with contrast prior to drug release or subsequent to drug release and generally offer no quantifiable signal values. Thus none of the hitherto clinically used imaging methods is suitable to provide quantitative information over the entire treatment process when using stimuli-responsive carriers for drug delivery.

There is, hence a need for an efficient, and reliable method of imaging-guided drug delivery, which provides quantitative information over the entire treatment process as well as for means to carry out such a method.

### OBJECTS AND SUMMARY OF THE INVENTION

The present invention addresses this need and provides means and methods for Magnetic Particle Imaging (MPI) guided drug delivery via stimuli-responsive carriers. The above objective is in particular accomplished by a composition comprising a shell structure forming a cavity, wherein said shell structure comprises a drug and wherein said composition is associated with at least one contrast agent; wherein said shell structure is capable of releasing its contents into the exterior upon the application of an external stimulus and wherein said contrast agent comprises magnetic particles which are capable of being detected by Magnetic Particle Imaging (MPI), wherein at least more than 5% (w/w) of the magnetic particles comprised in said contrast agent have a magnetic moment of at least 10⁻¹⁸ m²A, wherein said magnetic particles are preferably composed of Fe, Co, Ni, Zn or Mn or alloys thereof or oxides of any of these, more preferably composed of Fe₂O₃ or Fe₃O₄.

Such compositions combine the advantageous properties of stimuli-responsive carriers, i.e. the possibility of releasing substances, in particular drugs, at predetermined locations after the application a suitable signal, stimulus or action, and the advantageous properties of the Magnetic Particle Imaging (MPI) technology which allows the direct detection of the spatial distribution of magnetic nanoparticles from a non-linear remagnetization analysis resulting in a high sensitivity and resolution. It could, in particular, be shown that an MPI imaging signal generated by a contrast agent capable of being detected by MPI is not affected by the incorporation of the contrast agent into the composition or carrier. Furthermore, the signals could be shown to remain unaffected upon the release of the contrast agent from the carrier. Thus, in contrast to MRI-based methods, such compositions can be traced quantitatively using Magnetic Particle Imaging prior to drug release, as well as the further distribution of the composition's content can be followed subsequent to drug release.

In a preferred embodiment of the present invention at least more than 5% (w/w) of the magnetic particles have a remagnetization time of less than 10 milliseconds per particle.

In a further preferred embodiment of the present invention the contrast agent is associated with the outside or with internal portions of the shell structure or is associated with the drug or is embedded within the cavity of the shell structure.

In another preferred embodiment of the present invention the shell structure constitutes a liposome, a polymersome, a nanocapsule or any mixtures thereof. In a particularly preferred embodiment the shell comprises a thermo- or pressure-sensitive material.

In a further preferred embodiment of the present invention the external stimulus as mentioned above is capable of forming pores and/or of decomposing the shell structure.

In another preferred embodiment of the present invention the external stimulus is a temperature-increase, a temperature-decrease, a pressure-increase and/or a pressure-decrease.

In another aspect the present invention relates to the use of (i) a composition comprising a shell structure forming a cavity, wherein the shell structure comprises a drug and wherein the composition is associated with at least one contrast agent, wherein the contrast agent is capable of being detected by Magnetic Particle Imaging (MPI) and wherein the shell structure is capable of releasing its contents into the exterior upon the application of an external stimulus; or (ii) a composition as defined herein above, as a carrier for a controlled delivery of a drug.

In another preferred embodiment of the present invention the controlled delivery comprises a detection or localization using MPI. In a further alternative embodiment of the present invention, the controlled delivery comprises a detection or localization using MPI and Magnetic Resonance Imaging (MRI).

In another preferred embodiment of the present invention the use of (i) a composition comprising a shell structure forming a cavity, wherein the shell structure comprises a drug and wherein the composition is associated with at least one contrast agent, wherein the contrast agent is capable of being detected by Magnetic Particle Imaging (MPI) and wherein the shell structure is capable of releasing its contents into the exterior upon the application of an external stimulus; or (ii) a composition as defined herein above, as a carrier for a controlled delivery of a drug as defined herein above, wherein the controlled release further comprises the release of the content(s) of the shell structure via the application of an external stimulus. In a particularly preferred embodiment of the present invention the external stimulus is a temperature-increase, a temperature-decrease, a pressure-increase and/or a pressure-decrease stimulus.

In a further aspect the present invention relates to a method of data acquisition for the control of a drug delivery process comprising the detection or localization via MPI of (i) a composition comprising a shell structure forming a cavity, wherein said shell structure comprises a drug and wherein said composition is associated with at least one contrast agent, wherein said contrast agent is capable of being detected by MPI and wherein said shell structure is capable of releasing its contents into the exterior upon the application of an external stimulus; or (ii) a composition as defined herein above, before, during and/or after the application of an external stimulus releasing the content(s) of the shell structure.

In a further preferred embodiment of the present invention the method of data acquisition for the control of a drug delivery process comprises detection or localization via MPI and additionally via MRI. In a further embodiment of the present invention the method of data acquisition for the control of a drug delivery process comprises detection or localization via MPI and additionally via MRI.

In a further preferred embodiment of the present invention the method of data acquisition for the control of a drug delivery process as defined herein above comprises as additional step the release of the content(s) of said shell structure via the application of an external stimulus. In a particularly preferred embodiment of the present invention the external stimulus is a temperature-increase, a temperature-decrease, a pressure-increase and/or a pressure-decrease stimulus.

In a further aspect the present invention relates to a composition comprising a shell structure forming a cavity, wherein said shell structure comprises a drug and wherein said composition is associated with at least one contrast agent, wherein said contrast agent is capable of being detected by MPI and wherein said shell structure is capable of releasing its contents into the exterior upon the application of an external stimulus; or a composition as defined herein above for treating a pathological condition.

In a preferred embodiment of the present invention wherein the drug is to be administered by the application of a stimulus, wherein the stimulus is conveyed via a local heat system, via an electrical field, via a magnetic field, via focused ultrasound radiation and/or via radio frequency radiation, leading to the release of the drug from the shell structure into the exterior.

In another preferred embodiment of the present invention the composition is detectable or localizable with MPI. In a further embodiment of the present invention the composition is detectable or localizable with MPI and MRI.

### BRIEF DESCRIPTION OF THE DRAWINGS

FIG. 1 Schematic representation of the relative signal intensity in different imaging modalities in the process of drug delivery from a carrier encapsulating a respective agent.

FIG. 2 Schematic representation of the microencapsulation vesicle (MCV) technique used for the preparation of liposomes.

FIG. 3 Diagram showing UV-Vis absorption (hatched) and dynamic light scattering counts (cross-hatched) as a function of the elution volume.

FIG. 4 Image showing the result of an agarose gel electrophoresis (3% agarose gel, ethidium bromide-staining) of fractions collected after gel permeation chromatography of a DNA-loaded solution. The left line (A) corresponds to a reference DNA solution. Other lines are labeled according to the respective elution volume in mL.

FIG. 5 Image showing the result of an agarose gel electrophoresis (3% agarose gel, ethidium bromide-staining) of a DNA-loaded liposome solution before heating (A) and after heating for 30 minutes at 50° C. (B).

FIG. 6 Thermogram of a DNA/Resovist-loaded liposome solution subjected to a heating/cooling cycle comprised between 20° C. and 60° C. at a heating and cooling rate of 15° C./min.

FIG. 7 Image showing the result of an agarose gel electrophoresis (3% agarose gel, ethidium bromide-staining) of a DNA/Resovist-loaded liposome solution heated at 50° C. for different heating times. Line A represents a DNA ladder reference sample, Line B a herring sperm DNA reference solution, lines labelled 0 to 30 correspond to different heating times in minutes of the DNA/Resovist-loaded liposome sample.

FIG. 8 Illustration of ³¹P NMR spectra of DNA/Resovist loaded liposomes before (lower spectrum) and after (upper spectrum) heating to 55° C.

FIG. 9 Diagram showing the temperature dependence of the R₁ of DNA/Resovist-loaded liposomes measured during a positive temperature gradient (dots), a negative temperature gradient (up-triangles), a second positive temperature gradient (down-triangles), and a second negative temperature gradient (crosses).

FIG. 10 Cryo-TEM images of Resovist/DNA-loaded thermosensitive liposomes prepared using the MCV method before heating (A), after heating for 1 min at 50° C. (B), and after heating for 30 min at 50° C. (C). The black spots are Resovist particles. The scale bar represents 200 nm.

FIG. 11 Diagram showing an MPS signal (as a function of frequency) of DNA/Resovist-loaded thermosensitive liposomes before heating, and after 1 min, 4 min, and 30 min of heating at 50° C.

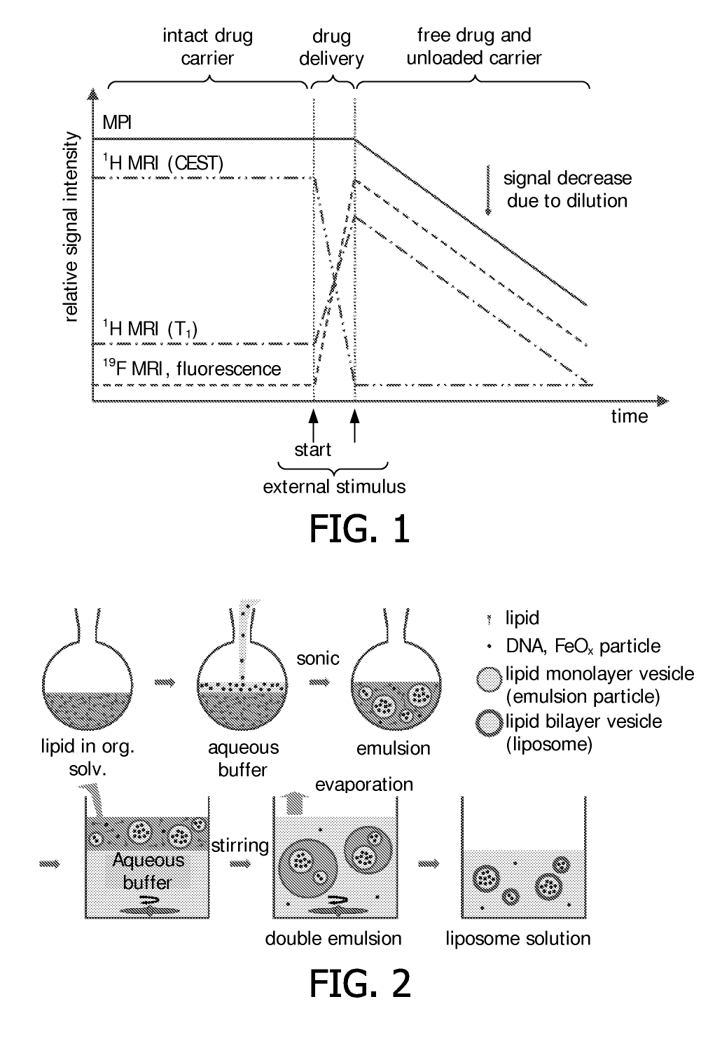

### DETAILED DESCRIPTION OF THE EMBODIMENTS

The present invention relates to means and methods for Magnetic Particle Imaging (MPI) guided drug delivery via stimuli-responsive compositions or carriers.

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In case the terms “first”, “second”, “third” or “(a)”, “(b)”, “(c)”, “(d)” etc. relate to steps of a method or use there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As has been set out above, the present invention concerns in one aspect a composition comprising a shell structure forming a cavity, wherein shell structure comprises a drug and wherein said composition is associated with at least one contrast agent; wherein said shell structure is capable of releasing its contents into the exterior upon the application of an external stimulus and wherein said contrast agent comprises magnetic particles which are capable of being detected by Magnetic Particle Imaging (MPI), wherein at least more than 5% (w/w) of the magnetic particles comprised in said contrast agent have a magnetic moment of at least 10⁻¹⁸ m²A, wherein said magnetic particles are preferably composed of Fe, Co, Ni, Zn or Mn or alloys thereof or oxides of any of these, more preferably composed of Fe₂O₃ or Fe₃O₄.

The term “shell structure” as used herein refers an envelope like structure, which is typically comprised of small units or entities which have identical or similar chemical, physical and/or biological properties. Furthermore, the envelope-like structure forms a cavity, i.e. excludes the exterior environment from the interior and hence serves as a boundary between exterior and interior environments, conditions etc. Shell structures according to the present invention may preferably be composed of a hydrophobic layer. The layer may be a monolayer or a bilayer. The sides of a bilayer structure may have different properties and/or be composed of different shell-forming units. Preferably, both sides will comprise hydrophobic tail structures pointing towards the interior of the shell structure or membrane. The shell structure may have a multilamellar form or a unilamellar form, constituting, for example, a small or large multilamellar vesicle, a small unilamellar vesicle or a large unilamellar vesicle. The shell structure may have any suitable form or dimension, e.g. the shell structure may be spherical, it may be elliptical, it may be circular or pear-shaped, dumb-bell like, flattened, pyramidal etc. The shell structure may preferably be capable of self-assembly.

In a typical embodiment of the present invention the shell forming units may be comprised of hydrophobic tails and hydrophilic heads. The interior or cavity of the shell structure may preferably constitute a hydrophilic environment, e.g. an aqueous solution. Alternatively the cavity of the shell structure may be comprised of a hydrophilic environment. The environment of the cavity of the shell structure may comprise the same environmental conditions as the exterior, or different environmental conditions. The term “environmental condition” as used herein refers to pH, the concentration of organic or inorganic ions, the presence of one or more salts, the presence of osmotic pressure etc. For instance, the pH in the cavity of the shell structure may be lower, identical or higher than the pH at the exterior, there can be osmotic pressure in the shell structure or there may be osmotic equilibrium etc.

In addition to shell-forming units, the shell may comprise further elements which provide additional functions. Examples of such additional elements are targeting entities, which allow an interaction and/or recognition of the shell structure by compatible elements, or stabilizing or destabilizing elements, which modify the chemical, physical and/or biological properties of the shell structure. These elements are typically present at the outside or outer surface of the shell structure and may or may not protrude into the interior of the shell structure and/or the cavity of the shell structure. Particularly preferred are elements which allow a targeting of the shell structure to specific tissue types, specific organs, cells or cell types or specific parts of the body, in particular the animal or human body. For example, the presence of targeting entities may lead to a targeting of the shell structures and thus, the entire composition, to organs like liver, kidney, lungs, heart, pancreas, gall, spleen, lymphatic structures, skin, brain, muscles etc. Alternatively, the presence of targeting entities may lead to a targeting to specific cell types, e.g. cancerous cells which express an interacting or recognizable protein at the surface. In a preferred embodiment of the present invention the shell structure may comprise proteins or peptides or fragments thereof, which offer an interaction surface at the outside and/or inside of the shell structure. Examples of such protein or peptide elements are ligands which are capable of binding to receptor molecules, receptor molecules, which are capable of interacting with ligands or other receptors, antibodies or antibody fragments or derivatives thereof, which are capable of interacting with their antigens, or avidin, streptavidin, neutravidin, lectins. Also envisaged by the present invention is the presence of binding interactors like biotin, which may, for example be present in the form of biotinylated compounds like proteins or peptides or shell structure units etc. or may be present within or at the outside of the shell structure itself. The shell structure may also comprise vitamins or antigens capable of interacting with compatible integrators, e.g. vitamin binding protein or antibodies etc., which can be either present at the surface of the shell structure and/or protrude into the shell structure and/or into the cavity of the shell structure.

The shell structure may also be covered by additional compounds, preferably by compounds which increase its stability and/or circulatory life, influence its biodistribution, modify its immunological behavior etc. Examples of such coatings include the presence of carbohydrate molecules, preferably the presence of glycosylation pattern, more preferably of biologically relevant glycosylation pattern typical for tissue or cell types as known to the person skilled in the art, or the presence of PEG (polyethylene glycol) at the outer layer or the outside of a shell structure. Particularly preferred is the use of polyethylene glycol 2000. Even more particularly preferred is the use of oligoglycerol (OG) groups. An example for the use of OG-modified thermosensitive liposomes is Lindner et al. 2008, Journal of Controlled Release, 125, 112-120.

Typical sizes of shell structures are from between about 30 nm and about 1000 nm. Preferred are sizes are between about 50 nm and about 400 nm.

The term “shell structure comprising a drug” as used herein means that a drug may be present in the cavity of the shell structure, at the surface of the shell structure, in the shell forming boundary region between the outside and the inside, e.g. the mono- or bilayer boundary itself, or at one or more of these compartments at the same time, e.g. stretching from the outside over the boundary section into the cavity of the shell structure, or stretching from the boundary section into the cavity of the shell structure, or stretching from the boundary section into the exterior. The drug may additionally be modified according to one or more of the modifications mentioned herein above, e.g. glycosylated, biotinylated, coated with PEG etc. Alternatively, a drug may be chemically or biologically modified in order to be capable of being present at the surface, the boundary region or the cavity of the shell structure. The drug may be present as monomer, as oligomer or as polymer. The presence may be adjusted in accordance with the osmotic situation, the charge of the shell structure or any other suitable parameter known to the person skilled in the art. In addition to a drug any suitable accessory molecule known to the person skilled in the art may be comprised in the shell structure, e.g. a stabilizing molecule, an adjuvant, an inhibitor of degrading enzymes, a charge stabilizer, a structure stabilizer, a salt, a buffer, an anti-oxidant, a chelating agent, a dye, e.g. a fluorescent dye, an imaging compound etc.

The term “drug” as used herein refers to any physical, chemical or biological substance which may be used in the treatment, cure, prophylaxis, prevention, or diagnosis of a pathological condition, e.g. a disease or disorder, or which may be used to otherwise enhance physical, psychical or mental well-being. The term also refers to substances which are of cosmetic use, which serve alimentary purposes or any combination of these aspects. In a preferred embodiment the term refers to bio-active agents. The term “bio-active agent” as used herein relates to biologically active agents including therapeutic drugs, endogenous molecules, and pharmacologically active agents, e.g. antibodies; nutritional molecules; cosmetic agents; diagnostic agents; and additional contrast agents for imaging. Also encompassed are active agents including pharmacologically acceptable salts of active agents.

Examples of drugs comprise nucleic acids such as polynucleotides, antisense nucleotides (gene therapy agents), RNA molecules, DNA molecules, siRNA molecules, miRNAs etc, carbohydrates, proteins or peptides, small molecules, lipids, lipopolysaccharides, non-peptide or non-protein drugs. It is possible within the scope of the present invention to incorporate drugs of a polymeric nature, but also to incorporate drugs of a relatively small molecular weight of less than 1500 g/mol, or even less than 500 g/mol.

Accordingly, bioactive agents envisaged in the context of the present invention include any compound with therapeutic or prophylactic effects. It can be a compound that affects or participates in tissue growth, cell growth, cell differentiation, a compound that is able to invoke a biological action such as an immune response, or a compound that can play any other role in one or more biological processes. A non-limiting list of examples includes antimicrobial agents (including antibacterial, antiviral agents and anti-fungal agents), anti-viral agents, anti-tumor agents, thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antimetabolites, antiproliferatives (including anti-angiogenesis agents), anticancer chemotherapeutic agents, anti-inflammatory steroid or non-steroidal anti-inflammatory agents, immunosuppressive agents, growth hormone antagonists, growth factors, dopamine agonists, radiotherapeutic agents, extracellular matrix components, ACE inhibitors, free radical scavengers, chelators, antioxidants, anti polymerases, and photodynamic therapy agents.

Relatively small peptides may be referred to by the number of amino acids (e.g. di-, tri-, tetrapeptides). A peptide with a relatively small number of amide bonds may also be called an oligopeptide (up to 50 amino acids), whereas a peptide with a relatively high number (more than 50 amino acids) may be called a polypeptide or protein. In addition to being a polymer of amino acid residues, certain proteins may further be characterized by the so called quaternary structure, a conglomerate of a number of polypeptides that are not necessarily chemically linked by amide bonds but are bonded by forces generally known to the skilled person, such as electrostatic forces and van-der-Waals forces. The term peptides, proteins or mixtures thereof as used herein is to include all above mentioned possibilities. Usually, the protein and/or peptide are selected on the basis of its biological activity. In a particular embodiment, the protein or peptide may be a growth factor.

Other examples of peptides or proteins or entities comprising peptides or proteins, which may advantageously be comprised in the shell structure include, but are not limited to, immunogenic peptides or immunogenic proteins, which include, but are not limited to, the following:

Toxins such as diphtheria toxin or tetanus toxin.

Viral surface antigens or parts of viruses such as adenoviruses, Epstein-Barr Virus, Hepatitis A Virus, Hepatitis B Virus, Herpes viruses, HIV-1, HIV-2, HTLV-III, Influenzaviruses, Japanese encephalitis virus, Measles virus, Papilloma viruses, Paramyxoviruses, Polio Virus, Rabies, Virus, Rubella Virus, Vaccinia (Smallpox) viruses and Yellow Fever Virus.

Bacterial surface antigens or parts of bacteria such as Bordetella pertussis, Helicobacter pylori, Clostridium tetani, Corynebacterium diphtheria, Escherichia coli, Haemophilus influenza, Klebsiella species, Legionella pneumophila, Mycobacterium bovis, Mycobacterium leprae, Mycrobacterium tuberculosis, Neisseria gonorrhoeae, Neisseria meningitidis, Proteus species, Pseudomonas aeruginosa, Salmonella species, Shigella species, Staphylococcus aureus, Streptococcus pyogenes, Vibrio cholera or Yersinia pestis.

Surface antigens of parasites causing disease or portions of parasites such as Plasmodium vivax (malaria), Plasmodium falciparum (malaria), Plasmodium ovale (malaria), Plasmodium malariae (malaria), Leishmania tropica (leishmaniasis), Leishmania donovani), leishmaniasis), Leishmania branziliensis (leishmaniasis), Trypanosoma rhodescense (sleeping sickness), Trypanosoma gambiense (sleeping sickness), Trypanosoma cruzi (Chagas' disease), Schistosoma mansoni (schistosomiasis), Schistosomoma haematobium (schistomiasis), Schistosoma japonicum (shichtomiasis), Trichinella spiralis (trichinosis), Stronglyloides duodenale (hookworm), Ancyclostoma duodenale (hookworm), Necator americanus (hookworm), Wucheria bancrofti (filariasis), Brugia malaya (filariasis), Loa loa (filariasis), Dipetalonema perstaris (filariasis), Dracuncula medinensis (filariasis), or Onchocerca volvulus (filariasis).

Immunoglobulins such as IgG, IgA, IgM, Antirabies immunoglobulins, and/or Antivaccinia immunoglobulins.

Antitoxin such as Botulinum antitoxin, diphtheria antitoxin, gas gangrene antitoxin or tetanus antitoxin.

Antigens which elicit an immune response against foot and mouth disease. Hormones and growth factors such as follicle stimulating hormone, prolactin, angiogenin, epidermal growth factor, calcitonin, erythropoietin, thyrotropic releasing hormone, insulin, growth hormones, insulin-like growth factors 1 and 2, skeletal growth factor, human chorionic gonadotropin, luteinizing hormone, nerve growth factor, adrenocorticotropic hormone (ACTH), luteinizing hormone releasing hormone (LHRH), parathyroid hormone (PTH), thyrotropin releasing hormone (TRH), vasopressin, cholecystokinin, and corticotropin releasing hormone; cytokines, such as interferons, interleukins, colony stimulating factors, and tumor necrosis factors: fibrinolytic enzymes, such as urokinase, kidney plasminogen activator; and clotting factors, such as Protein C, Factor VIII, Factor IX, Factor VII or Antithrombin III.

Examples of other proteins or peptides are albumin, atrial natriuretic factor, renin, superoxide dismutase, alpha 1-antitrypsin, lung surfactant proteins, bacitracin, bestatin, cyclosporine, delta sleep-inducing peptide (DSIP), endorphins, glucagon, gramicidin, melanocyte inhibiting factors, neurotensin, oxytocin, somostatin, terprotide, serum thymide factor, thymosin, DDAVP, dermorphin, Met-enkephalin, peptidoglycan, satietin, thymopentin, fibrin degradation product, des-enkephalin-alpha-endorphin, gonadotropin releasing hormone, leuprolide, alpha-MSH or metkephamid.

Anti-tumor agents such as altretamin, fluorouracil, amsacrin, hydroxycarbamide, asparaginase, ifosfamid, bleomycin, lomustin, busulfan, melphalan, chlorambucil, mercaptopurin, chlormethin, methotrexate, cisplatin, mitomycin, cyclophosphamide, procarbazin, cytarabin, teniposid, dacarbazin, thiotepa, dactinomycin, tioguanin, daunorubicin, treosulphan, doxorubicin, tiophosphamide, estramucin, vinblastine, etoglucide, vincristine, etoposid, vindesin or paclitaxel.

Antimicrobial agents comprising: antibiotics such as ampicillin, nafcillin, amoxicillin, oxacillin, azlocillin, penicillin G, carbenicillin, penicillin V, dicloxacillin, phenethicillin, floxacillin, piperacillin, mecillinam, sulbenicillin, methicillin, icarcillin, mezlocillin. Cephalosporins such as cefaclor, cephalothin, cefadroxil, cephapirin, cefamandole, cephradine, cefatrizine, cefsulodine, cefazolin, ceftazidim, ceforanide, ceftriaxon, cefoxitin, cefuroxime, cephacetrile, latamoxef, or cephalexin. Aminoglycosides such as amikacin, neomycin, dibekacyn, kanamycin, gentamycin, netilmycin or tobramycin. Macrolides such as amphotericin B, novobiocin, bacitracin, nystatin, clindamycin, polymyxins, colistin, rovamycin, erythromycin, spectinomycin, lincomycin or vancomycin. Tetracyclines such as chlortetracycline, oxytetracycline, demeclocycline, rolitetracycline, doxycycline, tetracycline or minocycline. Other antibiotics such as chloramphenicol, rifamycin, rifampicin or thiamphenicol.

Chemotherapeutic agents such as the sulfonamides sulfadiazine, sulfamethizol, sulfadimethoxin, sulfamethoxazole, sulfadimidin, sulfamethoxypyridazine, sulfafurazole, sulfaphenazol, sulfalene, sulfisomidin, sulfamerazine, sulfisoxazole and trimethoprim with sulfamethoxazole or sulfametrole.

Urinary tract antiseptics such as methanamine, quinolones (norfloxacin, cinoxacin), nalidixic acid, nitro-compounds (nitrofurantoine, nifurtoinol) or oxolinic acid.

Drug for anaerobic infections such as metronidazole.

Drugs for tuberculosis such as aminosalicyclic acid, isoniazide, cycloserine, rifampicine, ethambutol, tiocarlide, ethionamide or viomycin.

Drugs for leprosy such as amithiozone, rifampicine, clofazimine, sodium sulfoxone or diaminodiphenylsulfone (DDS, dapsone).

Antifungal agents such as amphotericin B, ketoconazole, clotrimazole, miconazole, econazole, natamycin, flucytosine, nystatine and griseofulvin.

Antiviral agents such as aciclovir, idoxuridine, amantidine, methisazone, cytarabine, vidarabine or ganciclovir.

Chemotherapy of amebiasis such as chloroquine, iodoquinol, clioquinol, metronidazole, dehydroemetine, paromomycin, diloxanide, furoatetimidazole and emetine.

Anti-malarial agents such as chloroquine, pyrimethamine, hydroxychloroquine, quinine, mefloquine, sulfadoxine/pyrimethamine, pentamidine, sodium suramin, primaquine, trimethoprim or proguanil.

Anti-helminthiasis agents such as antimony potassium tartrate, niridazole, antimony sodium dimercaptosuccinate, oxamniquine, bephenium, piperazine, dichlorophen, praziquantel, diethylcarbamazine, pyrantel parmoate, hycanthone, pyrivium pamoate, levamisole, stibophen, mebendazole, tetramisole, metrifonate, thiobendazole or niclosamide.

Anti-inflammatory agents such as acetylsalicyclic acid, mefenamic acid, aclofenac, naproxen, azopropanone, niflumic acid, benzydamine, oxyphenbutazone, diclofenac, piroxicam, fenoprofen, pirprofen, flurbiprofen, sodium salicyclate, ibuprofensulindac, indomethacin, tiaprofenic acid, ketoprofen or tolmetin.

Anti-gout agents such as colchicine or allopurinol.

Centrally acting (opoid) analgesics such as alfentanil, methadone, bezitramide, morphine, buprenorfine, nicomorphine, butorfanol, pentazocine, codeine, pethidine, dextromoramide, piritranide, dextropropoxyphene, sufentanil or fentanyl.

Local anesthetics such as articaine, mepivacaine, bupivacaine, prilocalne, etidocaine, procaine, lidocaine or tetracaine.

Drugs for Parkinson's disease such as amantidine, diphenhydramine, apomorphine, ethopropazine, benztropine mesylate, lergotril, biperiden, levodopa, bromocriptine, lisuride, carbidopa, metixen, chlorphenoxamine, orphenadrine, cycrimine, procyclidine, dexetimide or trihexyphenidyl.

Centrally active muscle relaxants such as baclofen, carisoprodol, chlormezanone, chlorzoxazone, cyclobenzaprine, dantrolene, diazepam, febarbamate, mefenoxalone, mephenesin, metoxalone, methocarbamol or tolperisone.

Corticosteroids comprising: Mineralocorticosteroids such as cortisol, desoxycorticosterone and fluorohydrocortisone. Glucocorticosteroids such as beclomethasone, betamethasone, cortisone, dexamethasone, fluocinolone, fluocinonide, fluocortolone, fluorometholone, fluprednisolone, flurandrenolide, halcinonide, hydrocortisone, medrysone, methylprednisolone, paramethasone, prednisolone, prednisone and triamcinolone (acetonide). Androgens comprising: androgenic steroids used in therapy such as danazole, fluoxymesterone, mesterolone, methyltestosterone, testosterone and salts thereof. Anabolic steroids used in therapy such as calusterone, nandrolone and salts thereof, dromostanolone, oxandrolone, ethylestrenol, oxymetholone, methandriol, stanozolol, methandrostenolone and testolactone. Antiandrogens such as cyproterone acetate. Estrogens comprising estrogenic steroids used in therapy such as diethylstilbestrol, estradiol, estriol, ethinylestradiol, mestranol or quinestrol. Anti-estrogens such as chlorotrianisene, clomiphene, ethamoxytriphetol, nafoxidine and tamoxifen. Progestins such as allylestrenol, desogestrel, dimethisterone, dydrogesterone, ethinylestrenol, ethisterone, ethynadiol diacetate, etynodiol, hydroxyprogesterone, levonorgestrel, lynestrenol, medroxyprogesterone, megestrol acetate, norethindrone, norethisterone, norethynodrel, norgestrel, and progesterone.

Thyroid drugs comprising: thyroid drugs used in therapy such as levothyronine and liothyronine. Anti-thyroid drugs used in therapy such as carbimazole, methimazole, methylthiouracil or propylthiouracil.

Preferred therapeutic agents are in the area of cancer (e.g. antitumor) and cardiovascular diseases.

Methods of preparing lipophilic drug derivatives which are suitable for shell structure formulations would be known to the person skilled in the art, e.g., from U.S. Pat. No. 5,534,499 describing covalent attachment of therapeutic agents to a fatty acid chain of a phospho lipid.

Drugs in the present invention can also be prodrugs. The present invention also envisages any suitable combination of drugs, e.g. a combination of any of the drugs mentioned herein above.

The term “capable of releasing its contents into the exterior” as used herein refers to the capability of the shell structure to be dissolved, disintegrated or opened up at least to a degree which allows the effusion of elements comprised in the cavity and/or the entire disintegration of the shell structure. The effusion may be partial or complete, i.e. a percentage of about 10, 20, 30, 40, 50, 60, 70, 80, 90 or up to 100% of the contents of the cavity may be provided to the exterior of the shell structure. The dissolving, opening or disintegration process of the shell structure may be permanently or reversible. In particular, if shell structure elements are used which are able to self assemble a reversible disintegration process may take place. Upon a reversible disintegration hollow shell structures without payload being present in its cavity or inside of the shell itself may remain. The disintegration or opening may further depend on the duration, type and form of stimulus. For example, a one time, timely limited stimulus may either lead to a non-reversible, permanent disintegration of the shell structure or to a reversible disintegration or opening of the shell structure which may return to its original form and/or size or to a different form and/or size but similar overall structure upon the termination of the stimulus. Preferably, a timely limited stimulus may lead to a timely limited opening of the shell structure allow the release of a portion of the contents of its cavity. The portion of the released contents may be proportional to or depend on the duration of the stimulus.

The term “external stimulus” as used herein refers to any change of condition at the localization of the composition or shell structure of the present invention, which is not originating from within the shell structure or composition and which is capable of provoking a release as defined herein above. Such a change of condition may be a change of one or more parameters like temperature, pressure, pH, ion concentration, fluidic movement, the change of a magnetic field, the change of an electrical field, the presence of destabilizing molecules etc. The stimulus, in order to be external, may be originating from the exterior of the shell structure, the exterior of the composition, the exterior of the tissue or organ the composition is localized, or the exterior of the entire body or organism. Preferably, the stimulus is provided by suitable apparatuses or devices, which may, for instance, be adapted to the physiological and/or biochemical conditions at the place of action. Particularly preferred stimuli are generated by high intensity focused ultrasound (HIFU), high intensity radiofrequency (RF) radiation or fast switching magnetic fields. These stimuli may lead to the generation of temperature changes, pressure changes or temperature- and pressure-changes. In a further preferred alternative a device capable of Magnetic Particle Imaging may be used for the generation of such a stimulus, e.g. by adjusting the intensity or amount of energy used.

The term “contrast agent” as used herein refers to any suitable contrast agent being capable of being detected by Magnetic Particle Imaging (MPI). Preferably, the term relates to an agent, which comprises or consists of at least one magnetic particle, more preferably a conjunction or number of different or identical magnetic particles, which are individually or as group detectable by Magnetic Particle Imaging.

The term “Magnetic Particle Imaging” or “MPI” as used herein refers to a technique that relies on the nonlinearity of the magnetization curves of ferromagnetic material and the fact that the particle magnetization saturates at a specific magnetic field strength. The remagnetization of magnetic contrast agents typically depends on parameters like the magnetic particle or material composition, its volume and its magnetic anisotropy, the particle size as a whole as well as the size of the magnetic core or of the magnetic cores, if the magnetic particle comprises more than one individual magnetic core, and its distribution etc. The term particularly refers to the technique of magnetic particle imaging as described above or derivable from Gleich et al., 2005, Nature, 435, 1214-1217 in several spatial dimensions, e.g. in a zero-dimension, in dimension one, in dimension two or in dimension three. An example of a zero-dimension Magnetic Particle Imaging is Magnetic Particle Spectroscopy (MPS), which typically provides the remagnetization signal without reconstructing images thereof. An example of a one-dimension Magnetic Particle Imaging is an acquisition method using a single-sided device as described in Sattel et al., 2009, Journal of Physics D: Applied Physics, 42, 1-5. An example of a two-dimension Magnetic Particle Imaging is an acquisition method that is feasible upon extension of the one-dimensional Magnetic Particle Imaging into a second dimension. An example of a three-dimension Magnetic Particle Imaging is the classical MPI.

The term “capable of being detected by Magnetic Particle Imaging” as used herein refers to the presence of one or more parameters in a contrast agent, which allows a detection, preferably a diagnostically suitable or high resolution detection of the contrast agent by MPI as described herein above. One such parameter is the identity of the contrast agent as at least one magnetic particle, preferably as a conjunction or number of different or identical magnetic particles. The conjunction may, for instance, comprise one, two, 3, 4, 5, 6, 7, 8, 9, 10, 15, 20 or up to 100 different magnetic particles. The term “different” as used herein refers to a difference in size, a difference in mass, a difference in magnetic moment, a difference in composition, a difference in magnetic anisotropy, a difference in remagnetization time etc. or any combination of these differences.

Preferably, a contrast agent according to the present invention may comprise at least more than 5% (w/w) of magnetic particles comprised in the contrast agent which is comprised in the composition according to the present invention, that have a magnetic moment of at least 10⁻¹⁸ m²A, more preferably of at least 2×10⁻¹⁸, 4×10⁻¹⁸, 6×10⁻¹⁸ or 8×10⁻¹⁸ m²A or even more preferably of at least 10⁻¹⁷ m²A. More preferably 6, 7, 8, 9, 10, 15, 20, 25, 30, 35, 40, 45, 50, 55, 60, 65, 70%, even more preferably 80, 90, 95% or even 100% (w/w) of the magnetic particles comprised in the contrast agent have a magnetic moment of at least 10⁻¹⁸ m²A. In another embodiment of the present invention 5% of the number of individual magnetic particles comprised in the contrast agent, which is comprised in the composition according to the present invention have a magnetic moment of at least 10⁻¹⁸ m²A. More preferably 6, 7, 8, 9, 10, 15, 20, 25, 30, 35, 40, 45, 50, 55, 60, 65, 70%, even more preferably 80, 90, 95% or even 100% of the number of individual magnetic particles comprised in the contrast agent which is comprised in the composition according to the present invention have a magnetic moment of at least 10⁻¹⁸ m²A. This parameter may be measured or tested according to any suitable method known to the person skilled in the art. Preferably a method as described in Kötitz et al, 1995, J. of Magnetism and Magnetic Materials, 149, 42-46 may be used. This method may also be combined with additional testings or assays known in the field of magnetic materials.

The size of a magnetic particle comprised in the contrast agent according to the present invention may vary between a diameter of about 5 nm and 50 nm. Preferably, the size of a magnetic particle is about 15, 20, 25, 30 or 35 nm. Most preferred is a diameter of >15 nm. In a preferred embodiment of the present invention at least more than 5% (w/w) of magnetic particles comprised in the contrast agent which is comprised in the composition according to the present invention have a size of about 5 nm to 50 nm, preferably of 15, 20, 25, 30 or 35 nm, more preferably of >15 nm. More preferably, 6, 7, 8, 9, 10, 15, 20, 25, 30, 35, 40, 45, 50, 55, 60, 65, 70%, even more preferably 80, 90, 95% or even 100% (w/w) of the magnetic particles comprised in the contrast agent have a size of about 5 nm to 50 nm, preferably of 15, 20, 25, 30 or 35 nm, more preferably of >15 nm. In another embodiment of the present invention 5% of the number of individual magnetic particles comprised in the contrast agent which is comprised in the composition according to the present invention have a size of about 5 nm to 50 nm, preferably of 15, 20, 25, 30 or 35 nm, more preferably of >15 nm. More preferably, 6, 7, 8, 9, 10, 15, 20, 25, 30, 35, 40, 45, 50, 55, 60, 65, 70%, even more preferably 80, 90, 95% or even 100% of the number of individual magnetic particles comprised in the contrast agent which is comprised in the composition according to the present invention have a size of about 5 nm to 50 nm, preferably of 15, 20, 25, 30 or 35 nm, more preferably of >15 nm. This parameter may be measured or tested according to any suitable method known to the person skilled in the art. Preferably a method as described in Kötitz et al, 1995, J. of Magnetism and Magnetic Materials, 149, 42-46 may be used. This method may also be combined with additional testings or assays known in the field of magnetic materials. Another preferred method is transmission electron microscopy. The use of transmission electron microscopy to measure particle sizes is well known to the person skilled in the art.

Alternatively, the remagnetization time of a magnetic particle comprised in the contrast agent according to the present invention may vary between about 12 and 0.1 milliseconds per particle, preferably between about 10 and 0.5 milliseconds per particle, more preferably be less than 10 or less than 8 milliseconds per particle. In a preferred embodiment of the present invention at least more than 5% (w/w) of magnetic particles comprised in the contrast agent which is comprised in the composition according to the present invention have a remagnetization time between about 12 and 0.1 milliseconds per particle, preferably between about 10 and 0.5 milliseconds per particle, more preferably less than 10 or less than 8 milliseconds per particle. More preferably 6, 7, 8, 9, 10, 15, 20, 25, 30, 35, 40, 45, 50, 55, 60, 65, 70%, even more preferably 80, 90, 95% or even 100% (w/w) of the magnetic particles comprised in the contrast agent have a remagnetization time between about 12 and 0.1 milliseconds per particle, preferably between about 10 and 0.5 milliseconds per particle, more preferably of less than 10 or less than 8 milliseconds per particle. In another embodiment of the present invention 5% of the number of individual magnetic particles comprised in the contrast agent which is comprised in the composition according to the present invention have a remagnetization time between about 12 and 0.1 milliseconds per particle, preferably between about 10 and 0.5 milliseconds per particle, more preferably of less than 10 or less than 8 less milliseconds per particle. More preferably 6, 7, 8, 9, 10, 15, 20, 25, 30, 35, 40, 45, 50, 55, 60, 65, 70%, even more preferably 80, 90, 95% or even 100% of the number of individual magnetic particles comprised in the contrast agent which is comprised in the composition according to the present invention have a remagnetization time between about 12 and 0.5 milliseconds per particle, preferably between about 10 and 0.5 milliseconds per particle, more preferably of less than 10 or less than 8 milliseconds per particle. This parameter may be measured or tested according to any suitable method known to the person skilled in the art. Preferably a method as described in Kötitz et al, 1995, J. of Magnetism and Magnetic Materials, 149, 42-46 may be used. This method may also be combined with additional testings or assays known in the field of magnetic materials.

One or more of these parameters may be present or given in one magnetic particle according to the present invention, e.g. a magnetic particle according to the present invention may show a magnetic moment as defined herein above and/or a size as defined herein above and/or a remagnetization time as defined herein above. In a specific embodiment of the present invention a magnetic particle according to the present invention may (i) have magnetic moment of at least 10⁻¹⁸ m²A, a size of >15 nm and show a remagnetization time of less than 10 or less than 8 milliseconds. Alternatively (ii), a magnetic particle according to the present invention may have magnetic moment of at least 10⁻¹⁸ m²A and a size of >15 nm. Alternatively (iii), a magnetic particle according to the present invention may have magnetic moment of at least 10⁻¹⁸ m²A and show a remagnetization time of less than 10 or less than 8 milliseconds. Alternatively (iv), a magnetic particle according to the present invention may have a size of >15 nm and show a remagnetization time of less than 10 or less than 8 milliseconds. In a further alternative of the present invention 5% (w/w) of magnetic particles comprised in the contrast agent which is comprised in the composition according to the present invention may show a combination of parameters as defined herein above in (i) to (iv). In a further alternative of the present invention 6, 7, 8, 9, 10, 15, 20, 25, 30, 35, 40, 45, 50, 55, 60, 65, 70%, even more preferably 80, 90, 95% or even 100% (w/w) of the magnetic particles comprised in the contrast agent which is comprised in the composition according to the present invention may show a combination of parameters as defined herein above in (i) to (iv). In yet another alternative of the present invention 5% of the number of individual magnetic particles comprised in the contrast agent which is comprised in the composition according to the present invention may show a combination of parameters as defined herein above in (i) to (iv). In yet another alternative embodiment 6, 7, 8, 9, 10, 15, 20, 25, 30, 35, 40, 45, 50, 55, 60, 65, 70%, even more preferably 80, 90, 95% or even 100% of the number of individual magnetic particles comprised in the contrast agent which is comprised in the composition according to the present invention may show a combination of parameters as defined herein above in (i) to (iv).

A magnetic particle according to the present invention may be composed of any suitable material known to the person skilled in the art. Preferably, the particle is composed of magnetic material, more preferably of Fe, Co, Ni, Zn, Mn etc. or chemical derivatives thereof. Typical derivatives which are preferable envisaged by the present invention are alloys or oxides of metals, e.g. alloys or oxides of Fe, Co, Ni, Zn or Mn or any combination thereof. Particularly preferred are oxides of iron, e.g. Fe₂O₃ or Fe₃O₄. Also encompassed by the present invention are magnetic particles composed of ferrite material or of doped materials, e.g. Co, Ni, Zn or Mn:FeₓO_{y}.

In a preferred embodiment of the present invention the contrast agent as mentioned herein above is associated with the outside or with internal portions of the shell structure as defined herein above, or is associated with a drug as defined herein above or is embedded within the cavity of said shell structure. The term “associated” as used herein refers to any perpetuation in terms of space sharing between the contrast agent and structures on the outside of the shell structure, within or between the shell constituting elements, i.e. in the boundary region between the outside and the inside of the shell structure or inside of the shell structure, i.e. in the cavity of the shell structure. The association may be a simple co-representation of entities in the same volume segment, e.g. if the contrast agent is embedded in the cavity of the shell structure. In such a situation there may be no binding or unity between the contrast agent and other comprised elements, in particular a drug according to the present invention. Alternatively, if a contrast agent is embedded or present in the shell structure, it may be bound, e.g. be covalently bound or bound by van-der-Waals-forces or ionic forces, with other compounds present in the cavity of a shell structure, e.g. one or more drugs as described herein above. Also envisaged by the present invention is a binding of the contrast agent to the building units of the shell structure, e.g. membrane constituting elements etc. A corresponding binding may be covalent, via van-der-Waals-forces or via ionic forces, preferably a covalent binding. In an alternative embodiment of the present invention, the contrast agent may be bound to the surface of the shell structure or to elements being anchored in said shell structure and being directed to the exterior, e.g. protein domains, peptides, sugar moieties, biotin, avidin etc. A corresponding binding may also be covalent, via van-der-Waals-forces or via ionic forces, preferably a covalent binding.

In another preferred embodiment of the present invention the shell structure as defined herein may be composed of one or more suitable amphipathic molecules as known to the person skilled in the art. Examples of such molecules are lipids, phospho lipids, hydrocarbon-based surfactants, cholesterol, glyco lipids, bile acids, saponins, fatty acids, synthetic amphipathic block copolymers, natural products like egg yolk phospho lipids etc. Particularly preferred are phospholipids and synthetic block copolymers. In a particularly preferred embodiment of the present invention a shell structure according to the present invention constitutes a liposome, a micelle, a polymersome, a nanocapsule or any mixtures thereof, more preferably any such structure comprising an amphipathic molecule as defined herein above.

The term “liposome” as used herein refers to a vesicle type which is typically made of lipids, in particular phospholipids, i.e. molecules forming a membrane like structure with a bilayer in aqueous environment. Preferred phospho lipids to be used in the context of liposomes include phosphatidylethanolamine, phosphatidylcho line, egg phosphatidyl-ethanolamine, dioleoylphosphatidylethanolamine. Particularly preferred are the phospholipids MPPC, DPPC, DPPE-PEG2000 or Liss Rhod PE. Liposomes typically comprise an aqueous and hydrophilic cavity, which may be used for the transport of correspondingly solvable compounds, e.g. hydrophilic drugs as defined herein above. The entrapment or packaging of a drug within liposomes of the present invention may be carried out using any conventional method in the art. Liposomes will typically be spherical. However, for use in the invention, such spherical carriers may be rendered aspherical. E.g. in the case of liposomes, this may be done by subjecting the liposomes to a dialysis process against a hypertonic buffer solution, hence a buffer solution with a higher osmolarity compared to the solution at the inside of the liposomes. The dialysis causes a net diffusion of water from the inside of the liposomes to the bulk solution. This reduces the total inner volume of the liposomes. Since the surface area of the liposomes remains constant, the volume reduction forces the liposomes to deform and to assume an aspherical shape, such as a disk shape, a cigar shape, or any other aspherical shape.

Lipososomes may be prepared by any suitable method known to the person skilled in the art, e.g. according to a formulation similar to the one described in U.S. Pat. No. 6,726,925. For liposome preparation preferably the microencapsulation vesicle (MCV) method may be used, utilizing water/oil/water (W/O/W) double emulsions, in which the drug load of the prepared liposomes is provided by the inner aqueous phase. This method is particularly suitable for preparing liposomes carrying hydrophilic drug molecules.

The term “micelle” as used herein refers to a vesicle type which is also typically made of lipids, in particular phosopholipids, which are organized in a monolayer structure. Micelles typically comprise a hydrophobic interior or cavity which may be used for the transport of correspondingly solvable compounds, e.g. hydrophobic drugs as defined herein above.

The term “nanocapsule” as used herein refers to a submicroscopic colloidal drug carrier system composed of an oily, an aqueous, or a gaseous core surrounded by a thin polymer membrane. Simplified they may consist of oil droplets in which lipophilic drugs may be solved. This oil core may be enclosed by a spherical polymer matrix. Nanocapsules may be generated according to any suitable technique known in the field, e.g. an interfacial polymerization of a monomer or the interfacial nanodeposition of a preformed polymer. A nanocapsule may have the form and consistency of a nanovesicle or of a nanosphere. A “nanosphere” includes but is not limited to a sphere which is not smaller than 5 nm. Nanospheres typically do not contain a cavity.

The term “polymersome” as used herein means a vesicle-type which is typically composed of block copolymer amphiphiles, i.e. synthetic amphiphiles that have an amphiphilicity similar to that of lipids. By virtue of their amphiphilic nature (having a more hydrophilic head and a more hydrophobic tail), the block copolymers are capable of self-assembling into a head-to-tail and tail-to-head bilayer structure similar to liposomes. Compared to liposomes, polymersomes have much larger molecular weights, with number average molecular weights typically ranging from 1000 to 100,000, preferably of from 2500 to 50,000 and more preferably from 5000 to 25000, are typically chemically more stable, less leaky, less prone to interfere with biological membranes, and less dynamic due to a lower critical aggregation concentration. These properties result in less opsonisation and longer circulation times. The terms “more hydrophilic” and “more hydrophobic” as used in the context of the ampohiphilic nature of the block copolymers are used in a relative sense. I.e., both can be either hydrophilic or hydrophobic, as long as the difference in polarity between the blocks is sufficient for the formation of polymersomes according to the present invention. In view of the creation of a cavity in which water may be incorporated, it is preferred for the more hydrophilic end of the polymer to be hydrophilic per se. Further, in view of the use as a drug carrier, it is desired that also hydrophobic drugs can be incorporated into the polymersomes. To this end, it is preferred that the hydrophobic end of the polymer is hydrophobic per se. The amphiphilic nature of the block copolymers is preferably realized in the form of a block copolymer comprising a block made up of more hydrophilic monomeric units (A) and a block made up of more hydrophobic units (B), the block copolymer having the general structure AₙBₘ, with n and m being integers of from 5 to 5000, preferably 10 to 1000, more preferably 10 to 500. It is also conceivable that one or more further units or blocks are built-in, e.g. a unit C with an intermediate hydrophilicity so as to yield a terpolymer having the general structure AₙCₚBₘ, with n and m being as defined above, and p being an integer of from 5 to 5000, preferably 10 to 1000, more preferably 10 to 500. Any of the blocks can itself be a copolymer, i.e. comprise different monomeric units of the required hydrophilic respectively hydrophobic nature. It is preferred that the blocks themselves are homopolymeric. Any of the blocks, in particular the more hydrophilic block, may bear charges. The number and type of charges may depend on the pH of the environment. Any combination of positive and/or negative charges on any of the blocks is contemplated by the present invention.

In view of the applicability in agents for drug delivery, it is preferred that the polymeric blocks are made of pharmaceutically acceptable polymers. Examples hereof are e.g. polymersomes as disclosed in US 2005/0048110. A polymersome-like structure may preferably be generated on the basis of a block copolymer, such as a block terpolymer, that intrinsically has the properties of forming a shell structure enclosing a cavity.

In combination with the use of contrast agents according to the present invention, the polymeric nature of the shell may be advantageously used by the incorporation of a variety of desired units. Thus, e.g., in order to achieve an increased contrast enhancement, the polymer itself can be rendered paramagnetic by the incorporation of ferromagnetic units, the enrichment of polymeric units with metal, metal alloys or methal oxides, or a combination thereof. An example of this approach is the enrichment by including iron or iron oxide-containing lipids into the liposome or polymersome structure, or the employment of iron or iron-oxide-containing copolymers. General references regarding metallopolymers may be derived from D. Wohrle, A. D. Pomogailo “Metal Complexes and Metals in Macromolecules” Wiley-VCH: Weinheim, 2003, and R. D. Archer “Inorganic and Organometallic Polymers” Wiley-VCH: New York, 2001. Preferably, the metallopolymer may comprise one type or different types of magnetic units with a high magnetic moment. The magnetic unit may be a part of the used lipids or the polymeric backbone or it may be linked to the polymeric chain via a linker connecting the polymer chain to a ligand encapsulating the metal.

Polymersomes may further be long circulating, as they are less prone to macrophage uptake. This property may be enhanced or modified by corresponding coatings and/or surface modifications.

In a further embodiment of the present invention the polymersomes may be semipermeable. The term “semipermeable” as used herein refers to the property of the shell structure to be selectively permeable, partially or differentially permeable. It indicates a structure that basically is closed in the sense that it is a not fully open wall, and preferably a mostly closed wall, (in this case a shell enclosing a cavity), that allows certain molecules or ions to pass through it by diffusion.

Polymersomes of the present invention may in a specific embodiment also be biodegradable and/or environment sensitive. This behavior may be controlled or influenced by the chemical structure of the copolymeric blocks.

Similar modifications as described in the context of polymersomes may also be carried out in the context of liposomes, micelles, or nanocapsules or of any other suitable shell structure known to the person skilled in the art.

Further details on shell structures, in particular polymersomes, and the manufacture thereof, may be derived from Antonietti et al., 2003, Adv. Mater., 15, No. 16 or from Soo et al., 2004, J. Pol. Sci., Part B: Polymer Physics, Vol. 42, 923-938.

Structure types and/or building blocks or lipsomes, micelles, polymersomes and/or nanocapsules may in a specific embodiment of the present invention be suitably mixed, e.g. in accordance with a desired size, target-type, hydrophobicity degree, pH, ion concentration etc. of the shell structure or composition.

In a further preferred embodiment of the present invention the shell structure according to the present invention, e.g. a liposome, micelle, polymersome or nanocapsule, may comprise environmental-sensitive material. The term “environmental sensitive material” as used herein refers to the material of the shell structure as a whole or building blocks comprised therein, which can be affected by external influences or stimuli. The affection may for instance be a change of the integrity of the shell structure, in particular a disintegration of the shell structure or a partial destruction of the shell structure. Such external influence or stimuli may include a change of temperature, in particular the application of heat, a change of pressure, pH, ion concentration, fluidic movement, the use of radio frequency radiation, the use of focused ultrasound radiation the change of a magnetic field, the change of an electrical field, radio frequency radiation the presence of destabilizing molecules etc. A typical example of such a stimulus is the typically decreased pH in tumor cells. Particularly preferred stimuli are generated by high intensity focused ultrasound (HIFU), high intensity radiofrequency (RF) radiation or fast switching magnetic fields. These stimuli may lead to the generation of temperature changes, pressure changes or temperature- and pressure-changes.

Furthermore, an environmental-sensitivity may be due to the biodegenerable or biodegradable nature of the shell structure. Thus, upon a controlled or anticipated biodegeneration or biodegradation of the shell structure the integrity of the structure may be weakened or destroyed leading to the release of drug molecules as described herein above.

Particularly preferred is the employment of a thermo- and/or pressure-sensitive material in the shell structure. The term “thermosensitive material” as used herein refers to a material in which the physical or chemical state of the shell structure is dependent on its temperature. Typically, a thermosensitive material can package a molecule of interest, e.g. a drug and will be intact at a normal body temperature (e.g. about. 37° C.) but can be destroyed, opened or disintegrated at any other, non-body temperature that can be tolerated by a subject. A thermally induced release of drugs, i.e. an opening or disintegration of the shell structure may preferably take place at a temperature of about 40° C., 41° C., 42° C., 43° C., 44° C., 45° C., 46° C., 47° C., 48° C. or 50° C., preferably at about 42° C. Thermosensitive material includes, inter alia, thermosensitive micro- and nanoparticles, thermosensitive polymersomes, thermosensitive liposomes or thermosensitive nanocapsules.

Thermosensitive liposomes may be composed of any one of MPPC, DPPC, DPPE-PEG2000 or Liss Rhod PE or any combination thereof. Particularly preferred is a ratio of 10 (MPPC):85 (DPPC):5 (DPPE-PEG2000). Further preferred is a ratio of 10 (MPPC):84.9 (DPPC):5 (DPPE-PEG2000):0.1 (Liss Rhod PE).

The required heat to raise the temperature of the thermosensitive material so as to promote the destruction, opening or disintegration of the thermosensitive material may be adapted to the tissue type, organ, distance between the surface and the target region etc. Heat may be applied in any physiologically acceptable way known to the person skilled in the art, preferably by using a focused energy source capable of inducing highly localized hyperthermia. The energy can be provided through, e.g., microwaves, ultrasound, magnetic induction, infrared or light energy.

The term “pressure-sensitive material” as used herein refers to a material in which the physical or chemical state of the shell structure is dependent on the pressure upon the material. Typically, a pressure-sensitive material can package a molecule of interest, e.g. a drug and will be intact at a normal pressure but can be destroyed, opened or disintegrated at any other pressure. The pressure may be produced either from within the shell structure or from the exterior. A change of local pressure may be generated in combination with the change of other parameters like the temperature. E.g. by increasing the local temperature also a pressure on the shell structure may be established which may lead to the disintegration or opening of the shell structure. The pressure modification may be provided by microwave, ultrasound or magnetic induction.

In a further embodiment of the present invention an external stimulus as defined herein above, e.g. a change of one or more parameters like temperature, pressure, pH, ion concentration, fluidic movement, the change of a magnetic field, the change of an electrical field, the use of radiofrequency radiation, the use of focused ultrasound radiation, the presence of destabilizing molecules etc. is capable of forming pores and/or of decomposing said shell structure. The term “forming pores” as used herein refers to the production of holes in the shell structure, preferably of a size which allows the efflux of drug molecules from the cavity to the exterior. Additionally, the efflux of contrast agents, e.g. magnetic particles according to the present invention may become possible via such pores. The pores may be transiently present or permanently, i.e. a pore may be closed after the stimulus is terminated, or it may stay open after the stimulus is terminated. The term “decomposing the shell structure” as used herein refers to the complete disintegration of the shell structure. The disintegration of the shell structure may lead to the release of the compounds comprised in the cavity, as well as the release of compounds, e.g. drug molecules, comprised in the membrane region or shell structure boundary itself. A decomposition of the shell structure is preferably irreversible, i.e. the shell structure may not be reformed or reshaped after the stimulus is terminated. In an alternative embodiment of the present invention the decomposition of the shell structure may be reversible, e.g. if building units are involved which are capable of self-assembly. A pore formation and the decomposition of the shell structure may also be combined, e.g. first pore may be formed, e.g. via one type of stimulus and subsequently the shell structure may be disintegrated entirely, e.g. via the application of a different type of stimulus. Such an approach may be employed to release two different types of drugs, e.g. one comprised in the cavity of a shell structure and a different drug comprised in the membrane or shell structure itself. The release processes may be separated with respect to their onset, i.e. first a pore formation may be induced and after a certain period of time, e.g. after several minutes, the decomposition of the shell structure may be induced.

In a preferred embodiment of the present invention the external stimulus is a temperature-increase, a temperature-decrease, a pressure-increase or a pressure-decrease. The term “increase” as used herein refers to an augmentation of a default or normal temperature or pressure by 1%, 2%, 3%, 4%, 5%, 6%, 7% or more. The term “decrease” as used herein refers to a diminishment of a default or normal temperature or pressure by 1%, 2%, 3%, 4%, 5% or more. The term “default or normal temperature” means a typical body temperature of e.g. about 37° C. in the case of human being. The typical body temperature may be different in other organisms, e.g. mammals as the person skilled in the art would know. The term “default or normal pressure” means a typical internal body pressure, e.g. a pressure in a blood vessel or artery or the pressure in an organ or tissue.

In a further aspect the present invention relates to the use of (i) a composition comprising a shell structure forming a cavity, wherein said shell structure comprises a drug and wherein said composition is associated with at least one contrast agent, wherein said contrast agent is capable of being detected by Magnetic Particle Imaging (MPI) and wherein said shell structure is capable of releasing its contents into the exterior upon the application of an external stimulus; or (ii) a composition as defined herein above, as a carrier for a controlled delivery of a drug. The term “controlled delivery of a drug” as used herein relates to the identification or detection of a location of a composition according to the present invention, and/or the detection of a movement of a composition according to the present invention, preferably via Magnetic Particle Imaging. A composition as mentioned herein, i.e. a shell structure comprising a drug, e.g. one or more of the drugs as mentioned herein above, may accordingly be employed for the transport and delivery of said drug(s) to a site of choice. The transport, sorting and/or the identification of such target sites or sites of choice may, for example, be influenced, controlled and/or triggered by the presence of interactor molecules comprised in the shell structure such as ligands, antibodies, antigens etc. or by the starting point, i.e. the localization of the composition at the beginning of the controlled delivery. Examples for such starting points are all major entry points of the human or animal body which may typically be used for the administration of pharmaceutical compositions, in particular for the application of contrast agents. Preferred are starting points in the cardiovascular system, e.g. in an artery or vein or any suitable blood vessel. Also preferred are starting points in organs or tissues of the animal or human body, e.g. the liver, the lungs, the spleen, the heart, the brain, muscle tissue etc. A composition according to the present invention may accordingly be transported and during this transport be monitored and controlled from the starting point to a target point, which may be either in proximity of the starting point or at a certain distance therefrom, e.g. in a distance of several cm, 10 cm, 50 cm, 75 cm etc. or even one or more than one meter. A composition according to the present invention may accordingly transverse the entire animal or human body or parts thereof, e.g. 10%, 20%, 30%, 40%, 50%, 60% etc. Such a transport or delivery may be monitored, tracked and the state and velocity of the transport process may be observed and registered by virtue of a contrast agent which is capable of being detected by Magnetic Particle Imaging (MPI), preferably a contrast agent comprising magnetic particles as defined herein above. The MPI signal, detectable e.g. via zero-dimensional MPI such as MPS or via classical three-dimensional MPI, may be received within an imaging voxel or a measuring volume of MPI and thus allows a quantitative determination of the contrast agent and, in consequence, of the composition(s) or shell structure(s) associated therewith. The signal may hence be used as a quantitative reflection or measurement of the overall contrast agent concentration within an imaging voxel. In particular, the “control” as used herein refers to the possibility of determining the absolute local contrast agent or magnetic particle concentration at a certain site, i.e. a quantitative determination of the contrast agent and thus composition and thus drug concentration at a defined spot.

A signal detection may be carried out at any suitable point in time, depending on the necessities of the approach, the capacity of the apparatus in use, the time management in a medical health care situation, the composition and/or size of the composition and/or the identity and nature of the contrast agent and/or the identity and properties of the drug etc. For instance, a signal may be detected between every 1 ms to 60 min, e.g. everyl ms, 2 ms, 5 ms, 10 ms, 20 ms, 30 ms, 50 ms, 100 ms, 200 ms, 500 ms, 700 ms, 1 sec, 5 sec, 10 sec, 20 sec, 30 sec, 40 sec, 50 sec, 1 min, 2 min, 5 min, 7 min, 10 min, 15 min, 20 min, 30 min etc. Signals may accordingly be recorded and analyzed with suitable devices, tools or programs known to the person skilled in the art.

Depending on the signal quality reception parameters of MPI may be adapted or changed in order to optimize or increase the signal quality. Accordingly obtained parameters or information may also be used as improved starting information for further, future applications.

In a further preferred embodiment of the present invention a controlled delivery comprises a detection or localization of a composition as defined herein above using MPI and additionally Magnetic Resonance Imaging (MRI). Contrast agents, in particular magnetic particles as defined herein above for MPI may accordingly also be used for Magnetic Resonance Imaging, which is typically based on the imaging of bulk water molecules being present at high concentrations throughout the body. In a specific embodiment of the present invention typically suitable contrast agents for MRI, e.g. a chemical shift contrast agent, e.g. the lipoCEST ¹H contrast agent, a gadolinium or manganese complex contrast agent, an MRI iron oxide particle contrast agent, or a ¹⁹F tracer, preferably in combination with a chemical shift contrast agent may be used in conjunction with a contrast agent detectable by MPI. One of more of these contrast agents may be comprised in a composition or shell structure as defined herein above. Also envisaged by the present invention is the use of compositions as defined herein above together with compositions comprising MRI contrast agents, which may also comprise one or more drugs as defined herein above. If these compositions are used together they should preferably have the same starting point within the organism, have additionally, in a further embodiment, the same or a similar size and/or same or a similar composition, i.e. are composed of the same shell structure building blocks, e.g. lipids, phospholipids, copolymers and/or the same or a similar mass etc., thus generating a similar or identical distribution pattern within a biological system, typically within the animal or human body.

In a further preferred embodiment of the present invention the use of a composition comprising a shell structure forming a cavity as mentioned herein above as a carrier for a controlled delivery of a drug further comprises the release of the content(s) of said shell structure via the application of an external stimulus. Accordingly, the surveillance of the transport or distribution and/or localization of a composition comprising a drug according to the present invention may be followed by the induction of the release of said drug into the environment once a predefined target region or site of choice has been reached, or, alternatively, after a predefined amount of time has passed. Alternatively or additionally, the release process itself may be observed or controlled via MPI based on the presence of a contrast agent in the cavity of the shell structure, a contrast agent being bound to the shell structure itself or a contrast agent being bound to the drug which is released from the shell structure. Depending on the exact localization and binding mode of the contrast agent a distribution of drug molecules within or next to the site of choice, a distribution of contrast agents per se or a distribution of shell structure remnants after the release may be detected.

Particularly preferred is a combination of MPI and MRI as mentioned herein above, wherein MPI may preferably be used upon the induction of a release from the composition to determine the absolute local particle concentration at a certain site and wherein MRI may preferably be used to visualize the drug release event as such. This approach may be carried out with only one type of contrast agent, which is capable of being detected by MPI and by MRI, e.g. a contrast agent comprising magnetic particles of different sizes or different magnetic moments or remagnetization times such as Resovist, or by combining typical MPI contrast agents and typical MRI contrast agents as defined herein above. Data and information obtained via MPI and/or MRI detection before, during and/or after the release of drugs may further be used as a feedback information for the control of the release itself, e.g. in order to increase the releasing stimulus, to slow down or shut down the releasing stimulus etc. Thus, if the MPI and in particular the MRI data obtained show a slow or incomplete release of the drug the applied stimulus may be changed, i.e. increased in time or intensity, or repeated one or more times. Alternatively, the release process may be stopped after, for example, about 10%, 20%, 30%, 40%, 50%, 60%, 70% or 80% of the drug was released, by terminating the stimulus which may preferably lead to a closing of pores or a reorganisation of the shell structure. Such partially released compositions may further be transported and distributed, which may be controlled and monitored by MPI. Subsequently, e.g. at a different location or site of choice the release process may be continued, leading again to a release or all or a part of the payload of a shell structure, e.g. about 10%, 20%, 30%, 40%, 50%, 60%, 70% and so on. This partial release may be repeated for one or more times, e.g. 2, 3, 4, 5 or up to 10 times.

An external stimulus may be a stimulus as described herein above, e.g. including a change of temperature, in particular the application of heat, a change of pressure, pH, ion concentration, fluidic movement, the use of radio frequency radiation, the use of focused ultrasound radiation, the change of a magnetic field, the change of an electrical field, the use of radiofrequency radiation and/or the presence of destabilizing molecules. A preferred external stimulus for the release of content(s) from the shell structure is a temperature-increase, a temperature-decrease, a pressure-increase and/or a pressure-decrease stimulus as defined herein above. Such a stimulus may be applied in any physiologically acceptable way known to the person skilled in the art, preferably by using a focused energy source capable of inducing highly localized hyperthermia. A pressure stimulus may be provided by any suitable technique known to the person skilled in the art, for example by microwave, ultrasound or magnetic induction etc. Particularly preferred stimuli are generated by high intensity focused ultrasound (HIFU), high intensity radiofrequency (RF) radiation or fast switching magnetic fields. These stimuli may lead to the generation of temperature changes, pressure changes or temperature- and pressure-changes.

In a further aspect the present invention relates to a method of data acquisition for the control of a drug delivery process comprising the detection or localization via MPI of (i) a composition comprising a shell structure forming a cavity, wherein said shell structure comprises a drug and wherein said composition is associated with at least one contrast agent, wherein said contrast agent is capable of being detected by MPI and wherein said shell structure is capable of releasing its contents into the exterior upon the application of an external stimulus; or (ii) a composition as defined herein above, before, during and/or after the application of an external stimulus releasing the content(s) of said shell structure. The term “data acquisition for the control of a drug delivery process” as used herein relates to the accumulation of information with regard to the location and whereabouts of a composition according to the present invention, and/or the movement of a composition according to the present invention, preferably via Magnetic Particle Imaging. A composition according to the present invention which transverses the entire animal or human body or parts thereof, e.g. 10%, 20%, 30%, 40%, 50%, 60% etc. may be monitored, tracked and the state and velocity of the transport process may be observed and registered by virtue of a contrast agent which is capable of being detected by Magnetic Particle Imaging (MPI), preferably a contrast agent comprising magnetic particles as defined herein above. The MPI signal, detectable e.g. via zero-dimensional MPI such as MPS or via classical three-dimensional MPI, may be received within an imaging voxel or a measuring volume of MPI and thus allows a quantitative determination of the contrast agent and, in consequence, of the composition(s) or shell structure(s) associated therewith. The signal may hence be used as a quantitative reflection or measurement of the overall contrast agent concentration within an imaging voxel, i.e. as data input for the definition of the composition or particle localization. In particular, the “control” as used herein refers to the possibility of determining the absolute local contrast agent or magnetic particle concentration at a certain site, i.e. a quantitative determination of the contrast agent and thus composition and thus drug concentration at a defined spot, preferably within a biological system, e.g. the animal or human body. A “drug delivery process” as used herein means at least one step of the following sequence of steps or actions comprising the introduction of a composition according to the present invention into a biological system, e.g. an animal or human body, the distribution or transport of the composition within said biological system and the arrival at a predefined area, zone, organ, tissue, cell layer, structure etc. of the biological system or body. The localization the composition and the concentration of contrast agents in said composition may particularly be monitored before the application of an external stimulus, i.e. up to the arrival at the site of choice and/or during the application of an external stimulus and/or after the application of an external stimulus, as described herein above. Each of these steps may be monitored, recorded, analyzed, and manipulated with MPI. Accordingly obtained information may be used for the decision of drug release or for diagnostic purposes.

In a specific embodiment of the present invention data on the localization and distribution of all or a certain percentage, e.g. 20%, 40%, 60%, 80% of all compositions within a biological system may be acquired. Accordingly obtained information may provide a picture of the movement and distribution of compositions starting from a common starting point as mentioned herein above. Alternatively, the information may be used to see whether compositions or associated drugs have been distributed systemically or to detect which percentage of the starting material, i.e. compositions at the starting point, has arrived at the destination of choice, e.g. a specific organ or tissue.

In a particularly preferred embodiment of the present invention the method of data acquisition for the control of a drug delivery process may include a detection or localization of the composition as defined herein above by MPI and MRI. A combined use of MPI and MRI as well as corresponding applications and advantages have been described herein above.

In a further embodiment of the present invention the method of data acquisition for the control of a drug delivery process comprises as additional step the release of the content(s) of the shell structure via the application of an external stimulus. The release of contents, in particular of a drug as defined herein above, may be coordinated with the data acquired during the detection and localization of the composition as described herein above, i.e. a release may be induced upon arrival at a target site or site of choice. Furthermore, a monitoring of the release process itself may be carried out, as well as a monitoring of the whereabouts of the composition, i.e. the shell structure, as described in the context of the uses of the present invention herein above. The stimulus to be employed may preferably be a temperature-increase, a temperature-decrease, a pressure-increase and/or a pressure-decrease stimulus as described herein above.

In a further embodiment the present invention relates to a method for the treatment and/or diagnosis of a pathological condition or a diseased organ or tissue, preferably of an animal or human body, comprising a controlled drug delivery process including the detection or localization via MPI of (i) a composition comprising a shell structure forming a cavity, wherein said shell structure comprises a drug and wherein said composition is associated with at least one contrast agent, wherein said contrast agent is capable of being detected by MPI and wherein said shell structure is capable of releasing its contents into the exterior upon the application of an external stimulus; or (ii) a composition as defined herein above, before, during and/or after the application of an external stimulus and the release of the content(s) of said shell structure via the application of an external stimulus, preferably a temperature-increase, a temperature-decrease, a pressure-increase and/or a pressure-decrease stimulus at a destination or localization of choice. The method comprises the introduction of a composition as described herein above at a suitable site, e.g. into the blood vessel, the monitoring of the movement of the composition and the release of the drug payload at a second site, preferably a release accompanied by monitoring actions of the release. The method may alternative comprise only the steps of monitoring of the movement of the composition and the release of the drug payload at a second site. The method may alternative comprise only the steps of introduction of a composition as described herein above at a suitable site, e.g. into the blood vessel, and the release of the drug payload at a second site.

In another aspect the present invention relates to a composition comprising a shell structure forming a cavity, wherein said shell structure comprises a drug and wherein said composition is associated with at least one contrast agent, wherein said contrast agent is capable of being detected by MPI and wherein said shell structure is capable of releasing its contents into the exterior upon the application of an external stimulus; or a composition as defined herein above for treating a pathological condition.

In further embodiment the present invention relates to a composition comprising a shell structure forming a cavity, wherein said shell structure comprises a drug and wherein said composition is associated with at least one contrast agent, wherein said contrast agent is capable of being detected by MPI and wherein said shell structure is capable of releasing its contents into the exterior upon the application of an external stimulus; or a composition as defined herein above for diagnosing a pathological condition.

In yet another embodiment the present invention relates to a method for the preparation of a pharmaceutical composition comprising a shell structure forming a cavity, wherein said shell structure comprises a drug and wherein said composition is associated with at least one contrast agent, wherein said contrast agent is capable of being detected by MPI and wherein said shell structure is capable of releasing its contents into the exterior upon the application of an external stimulus; or pharmaceutical composition as defined herein above for the treatment of a pathological condition.

In yet another embodiment the present the present invention relates to a method for the preparation of a diagnostic composition comprising a shell structure forming a cavity, wherein said shell structure comprises a drug and wherein said composition is associated with at least one contrast agent, wherein said contrast agent is capable of being detected by MPI and wherein said shell structure is capable of releasing its contents into the exterior upon the application of an external stimulus; or a diagnostic composition as defined herein above for the diagnosis of a pathological condition.

The term “pathological condition” as used herein refers to any type of disease, disorder, tissue or organ malfunction etc., which is targetable by a composition as defined herein above. For example, a pathological condition may be targetable if the diseased area or zone or the zone of malfunction is connected to the cardiovascular system, preferably if the cardiovascular system allows a passage of a composition or shell structure according to the present invention. Typical examples are all diseases which can be arrived if the composition is introduced into a blood vessel. Alternatively, a pathological condition may be targetable if the diseased area or zone or the zone of malfunction is connected to the lymphatic system, preferably if the cardiovascular system allows a passage of a composition or shell structure according to the present invention. In a further alternative, a pathological condition may be targetable if the diseased area or zone or the zone of malfunction is connected to the cerebrospinal fluid system, preferably if the cerebrospinal fluid system allows a passage of a composition or shell structure according to the present invention.

Pathological conditions which may be treated with a composition according to the present invention include, but are not limited to deficiencies or disorders of the immune system, e.g. the proliferation, differentiation, or mobilization (chemotaxis) of immune cells. Also included are deficiencies or disorders of hematopoictic cells. Examples of immunologic deficiency syndromes include, but are not limited to: blood protein disorders (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Further included are blood coagulation disorders (e.g. afibrinogenemia, factor deficiencies) or blood platelet disorders (e.g. thrombocytopenia), heart attacks (infarction) or strokes or pre-infarction conditions.

Further included are cardiovascular diseases, disorders, and conditions and/or cardiovascular abnormalities, such as arterio-arterial fistula, arteriovenous fistula, cerebral arteriovenous malformations, congenital heart defects, pulmonary atresia, and Scimitar Syndrome. Congenital heart defects include aortic coarctation, cor triatriatum, coronary vessel anomalies, crisscross heart, dextrocardia, patent ductus arteriosus, Ebstein's anomaly, Eisenmenger complex, hypoplastic left heart syndrome, levocardia, tetralogy of fallot, transposition of great vessels, double outlet right ventricle, tricuspid atresia, persistent truncus arteriosus, and heart septal defects, such as aortopulmonary septal defect, endocardial cushion defects, Lutembacher's Syndrome, trilogy of Fallot, ventricular heart septal defects. Cardiovascular diseases, disorders, and/or conditions also include heart disease, such as arrhythmias, carcinoid heart disease, high cardiac output, low cardiac output, cardiac tamponade, endocarditis (including bacterial), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve diseases, myocardial diseases, myocardial ischemia, pericardial effusion, pericarditis (including constrictive and tuberculous), pneumopericardium, postpericardiotomy syndrome, pulmonary heart disease, rheumatic heart disease, ventricular dysfunction, hyperemia, cardiovascular pregnancy complications, Scimitar Syndrome, cardiovascular syphilis, and cardiovascular tuberculosis. Arrhythmias include sinus arrhythmia, atrial fibrillation, atrial flutter, bradycardia, extrasystole, Adams-Stokes Syndrome, bundle-branch block, sinoatrial block, long QT syndrome, parasystole, Lown-Ganong-Levine Syndrome, Mahaimtype pre-excitation syndrome, Wolff-Parkinson-White syndrome, sick sinus syndrome, tachycardias, and ventricular fibrillation. Tachycardias include paroxysmal tachycardia, supraventricular tachycardia, accelerated idioventricular rhythm, atrioventricular nodal reentry tachycardia, ectopic atrial tachycardia, ectopic junctional tachycardia, sinoatrial nodal reentry tachycardia, sinus tachycardia, Torsades de Pointes, and ventricular tachycardia. Heart valve disease include aortic valve insufficiency, aortic valve stenosis, hear murmurs, aortic valve prolapse, mitral valve prolapse, tricuspid valve prolapse, mitral valve insufficiency, mitral valve stenosis, pulmonary atresia, pulmonary valve insufficiency, pulmonary valve stenosis, tricuspid atresia, tricuspid valve insufficiency, and tricuspid valve stenosis. Myocardial diseases include alcoholic cardiomyopathy, congestive v' cardiomyopathy, hypertrophic cardiomyopathy, aortic subvalvular stenosis, pulmonary subvalvular stenosis, restrictive cardiomyopathy, Chagas cardiomyopathy, endocardial fibroelastosis, endomyocardial fibrosis, Kearns Syndrome, myocardial reperfusion injury, and myocarditis. Myocardial ischemias include coronary disease, such as angina pectoris, coronary aneurysm, coronary arteriosclerosis, coronary thrombosis, coronary vasospasm, myocardial infarction and myo-cardial stunning Cardiovascular diseases also include vascular diseases such as aneurysms, angiodysplasia, angiomatosis, bacillary angiomatosis, Hippel-Lindau Disease, Klippel-Trenaunay-Weber Syndrome, Sturge-Weber Syndrome, angioneurotic edema, aortic diseases, Takayasu's Arteritis, aortitis, Leriche's Syndrome, arterial occlusive diseases, arteritis, enarteritis, polyarteritis nodosa, cerebrovascular diseases, disorders, and/or conditions, diabetic angiopathies, diabetic retinopathy, embolisms, thrombosis, erythromelalgia, hemorrhoids, hepatic veno-occlusive disease, hypertension, hypotension, ischemia, peripheral vascular diseases, phlebitis, pulmonary venoocclusive disease, Raynaud's disease, CREST syndrome, retinal vein occlusion, Scimitar syndrome, superior vena cava syndrome, telangiectasia, atacia telangiectasia, hereditary hemorrhagic telangiectasia, varicocele, varicose veins, varicose ulcer, vasculitis, and venous insufficiency. Aneurysms include dissecting aneurysms, false aneurysms, infected aneurysms, ruptured aneurysms, aortic aneurysms, cerebral aneurysms, coronary aneurysms, heart aneurysms, and iliac aneurysms. Arterial occlusive diseases include arteriosclerosis, intermittent claudication, carotid stenosis, fibromuscular dysplasias, mesenteric vascular occlusion, Moyamoya disease, renal artery obstruction, retinal artery occlusion, and thromboangiitis obliterans. Cerebrovascular diseases, disorders, and/or conditions include carotid artery diseases, cerebral amyloid angiopathy, cerebral aneurysm, cerebral anoxia, cerebral arteriosclerosis, cerebral arteriovenous malformation, cerebral artery diseases, cerebral embolism and thrombosis, carotid artery thrombosis, sinus thrombosis, Wallenberg's syndrome, cerebral hemorrhage, epidural hematoma, subdural hematoma, subaraxhnoid hemorrhage, cerebral infarction, cerebral ischemia (including transient), subclavian steal syndrome, periventricular leukomalacia, vascular headache, cluster headache, migraine, and vertebrobasilar insufficiency. Embolisms include air embolisms, amniotic fluid embolisms, cholesterol embolisms, blue toe syndrome, fat embolisms, pulmonary embolisms, and thromoboembolisms. Thrombosis include coronary thrombosis, hepatic vein thrombosis, retinal vein occlusion, carotid artery thrombosis, sinus thrombosis, Wallenberg's syndrome, and thrombophlebitis. Ischemia includes cerebral ischemia, ischemic colitis, compartment syndromes, anterior compartment syndrome, myocardial ischemia, reperfusion injuries, and peripheral limb ischemia. Vasculitis includes aortitis, arteritis, Behcet's Syndrome, Churg-Strauss Syndrome, mucocutaneous lymph node syndrome, thromboangiitis obliterans, hypersensitivity vasculitis, Schoenlein-Henoch purpura, allergic cutaneous vasculitis, and Wegener's granulomatosis.

Further included are autoimmune disorders such as Addison's Disease, hemolytic anemia, antiphospho lipid syndrome, rheumatoid arthritis, dermatitis, allergic encephalomyelitis, glomerulonephritis, Goodpasture's-Syndrome, Graves Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, or autoimmune inflammatory eye disease. Additionally included are allergic reactions and conditions, such as asthma (particularly allergic asthma) or other respiratory problems; as well as hyperproliferative disorders, including neoplasms, cancers or tumors, such as neoplasms, cancers or tumors located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital tract. Further examples of hyperproliferative disorders which may be treated are hypergammaglobulinemia, lymphoproliferative disorders, paraproteinemi as, purpura, sarcoidosis, Sezary Syndrome, Waldenstron's Macroglobulinermia, Gaucher's Disease, histiocytosis, and any other hyperproliferative disease, located in an organ system listed above.

Further included are neurodegenerative disease states, behavioral disorders, or inflammatory conditions which include, but are not limited to Alzheimer's Disease, Parkinson's Disease, Huntington's Disease, Tourette Syndrome, encephalitis, demyelinating diseases, peripheral neuropathies, trauma, congenital malformations, spinal cord injuries, ischemia, aneurysms, hemorrhages, schizophrenia, mania, dementia, paranoia, obsessive compulsive disorder, depression, panic disorder, learning disabilities, ALS, psychoses, autism, and altered behaviors, including disorders in feeding, sleep patterns, balance, or perception.

Additionally included are pathological conditions caused by infections. Viruses are one example of an infectious agent that can cause diseases or symptoms. Examples of viruses, include, but are not limited to the following DNA and RNA viral families: Arbovirus, Adenoviridae, Arenaviridae, Arterivirus, Bimaviridae, Bunyaviridae, Caliciviridae, Circoviridae, Coronaviridae, Flaviviridae, Hepadnaviridae (Hepatitis), Herpesviridae (such as Cytomegalovirus, Herpes Simplex, Herpes Zoster), Mononegavirus (e.g. Paramyxoviridae, Morbillivirus, Rhabdoviridae), Orthomyxoviridae (e.g. Influenza), Papovaviridae, Parvoviridae, Picornaviridae, Poxyiridae (such as Smallpox or Vaccinia), Reoviridae (e.g. Rotavirus), Retroviridae (HTLV-I, HTLV-II, Lentivirus), and Togaviridae (e.g. Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia.

Similarly, infections are included which are caused by bacterial or fungal agents such as Gram-Negative and Gram-positive bacterial families and fungi: Actinomycetales (e.g. Corynebacterium, Mycobacterium, Norcardia), Aspergillosis, Bacillaceae (e.g. Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g. Acinetobacter, Gonorrhea, Menigococcal), Pasteurellacea Infections (e.g. Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic˜infections (e.g. AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g. cellulitis, dermatocycoses), toxemia, urinary tract infections or wound infections.

Further included are infections or diseases caused by Escherichia coli, Staphylococcus epidermidis, Staphylococcus aureus, Enterococcus faecalis, Klebsiella pneumoniae, Pseudomonas aeruginosa, Enterococcus faecium, Streptococcus pneumoniae, Staphylococcus capitis, Klebsiella oxytoca, Streptococcus agalactiae, Proteus mirabilis, Staphylococcus cohnii, Staphylococcus haemolyticus, Acinetobacter baumannii, Enterococcus sp., Proteus vulgaris, Serratia marcescens, Staphylococcus warneri, Staphylococcus hominis, Streptococcus anginosus, Streptococcus mitis, Staphylococcus auricularis, Staphylococcus lentus, Streptococcus beta haem Group G, Streptococcus beta haem Group F, Streptococcus gordonii, Streptococcus Group D, Streptococcus oxalis, Streptococcus parasanguis, Streptococcus salivarius, Citrobacter freudii, Listeria monocytogenes, Micrococcus luteus, Acinetobacter junii, Bacillus cereus, Bacteroides caccae, Bacteroides uniformis, Bacteroides vulgatus, Clostridium perfringens, Corynebacterium pseudodiphtheriticum, Corynebacterium sp., Corynebacterium urealyticum, Fusiobacterium nucleatum, Micrococcus sp., Pasteurella multocida, Propionibacterium acnes, Ralstonia pickettii, Salmonella ser. Paratyphi B or Yersinia enterocditi.

Additionally, infections, diseases or symptoms are included which are caused by parasitic agents including, but not limited to Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas may be treated. These parasites can cause a variety of diseases or symptoms, including, but not limited to: Scabies, Trombiculiasis, eye infections, intestinal disease (e.g. dysentery, giardiasis), liver disease, lung disease, opportunistic infections (e.g. AIDS related), Malaria, pregnancy complications, and toxoplasmosis.

The treatment of a pathological condition as mentioned herein above may be combined with a further treatment approach, e.g. a classical treatment approach via oral, intravenous, nasal etc. application of a known medicament or pharmaceutical composition, e.g. medicament known to be effective for the treatment of the same disease or a connected pathological condition. For instance, a classical treatment approach may be used in order to treat a disease aspect systemically, whereas the composition according to the present invention may, at the same time or in the same treatment regimen, be used to treat the pathological condition locally.

In a particularly preferred embodiment of the present invention the composition, e.g. a pharmaceutical or diagnostic composition as defined herein, or drug comprised therein, is to be administered by the application of a stimulus leading to the release of said drug from said composition or shell structure into the exterior. The stimulus may preferably be an external stimulus, more preferably an increase or decrease of temperature, or an increase or decrease of pressure. Such a stimulus may be conveyed via any suitable technique or apparatus known to the person skilled in the art, e.g. via a local heat system, via an electrical field, via a magnetic field, via focused ultrasound radiation and/or via radiofrequency radiation. Particularly preferred stimuli are generated by high intensity focused ultrasound (HIFU), high intensity radiofrequency (RF) radiation or fast switching magnetic fields. These stimuli may lead to the generation of temperature changes, pressure changes or temperature- and pressure-changes.

Particularly preferred is the use of thermal stimuli, e.g. via local heat systems. The application of thermal stimuli may, in a further preferred embodiment, also be combined with an additional therapeutic approach based on local hyperthermia and the resulting therapeutic effects.

In a further preferred embodiment a composition according to the present invention, e.g. a pharmaceutical or diagnostic composition as defined herein, is detectable by MPI or a combination of MPI and MRI as described herein above. Accordingly the localization of the composition before, during and/or after the treatment may be determined or detected. Furthermore, the whereabouts of composition or shell structure remnants may be detected after the administration step. Such a detection may be useful for the assessment of biodynamic processes, the velocity of excretion, the assessment of corresponding toxicity parameters etc. Furthermore, the administration step, i.e. the release of the drug may be controlled and influenced or manipulated during the release, e.g. via feedback loop depending on the degree of release. This process itself may be controlled as has been described herein above.

In a further specific embodiment of the present invention the composition of the present invention may be used during ablation processes, e.g. the ablation of specific tissues, preferably cancerous tissues or organ portions. Such an ablation may be carried out by any suitable means known to the person skilled in the art, e.g. via high intensity focused ultrasound (HIFU) and/or MRI techniques. A composition of the present invention may accordingly be targeted and/or localized within a region which should be ablated. Subsequently, the composition may be trapped in the ablated tissue. Contrast agents present in the composition may either be kept inside the composition or be released, e.g. via stimuli or the ablation process itself. Due to the trapping of the contrast agent MPI may be used to demark the ablated region, i.e. to exactly define the ablated region. This information may be used for subsequent diagnostic or therapeutic steps, e.g. a repetition of the ablation process. Furthermore, the composition may comprise drugs which provide a therapeutical effect in accordance with the purpose of the ablation, e.g. chemotherapeutical agents or cancer medicaments may be localized and subsequently released.

The following examples and figures are provided for illustrative purposes. It is thus understood that the example and figures are not to be construed as limiting. The skilled person in the art will clearly be able to envisage further modifications of the principles laid out herein.

### EXAMPLES

### Example 1

### Preparation of DNA-Loaded Thermosensitive Liposomes

In a typical preparation of DNA-loaded thermosensitive liposomes 6.3 mg (8.5 μmol) of DPPC, 0.5 mg (1.0 μmol) of MPPC, 1.4 mg (0.5 μmol) of DPPE-PEG2000, and 25 μL of a 1 mg/mL solution of Liss Rhod PE in CHCl₃ were dissolved in CHCl₃ to obtain 1.0 mL of a CHCl₃ solution comprising a 10 mM concentration of lipids. DNA (herring sperm, Sigma-Aldrich) was dissolved in HEPES buffer (135 mM NaCl, 20 mM HEPES, pH 7.40), Resovist stock solution, or a mixture of both. 0.3 mL of the so obtained aqueous solution were mixed with the CHCl₃ solution to obtain a 0.3:1 water/oil (W/O) ratio. The constitution of the aqueous phase varied as described in the following Table 1:

TABLE 1
Composition of Examples 1-3.

| | c(DNA)/ | V(DNA)/ | c(Resovist)/ | V(Resovist)/ | |
|---|---|---|---|---|---|
| n | mg/mL | mL | mM | mL | label* |
| 1 | 30 | 0.30 | − | − | − |
| 2 | 30 | 0.30 | − | − | + |
| 3 | 30 | 0.15 | 0.50 | 0.15 | − |

*Sample was fluorescently labeled with Liss Rhod PE 0.1% in the lipid composition

The obtained mixture was sonicated using a QEX 600 sonication device, operated at a frequency of 20 kHz, an amplitude of 108 W, and a temperature of 20° C. for 5 minutes. The obtained W/O emulsion was poured into 8 mL of HEPES buffer solution, in a 25 mL Erlenmeyer flask. This mixture was stirred overnight at room temperature to allow for the slow evaporation of CHCl₃ yielding the crude liposome solution, which contained a fraction of not encapsulated Resovist particles. Those were removed by gel permeation chromatography (GPC) in the next step.

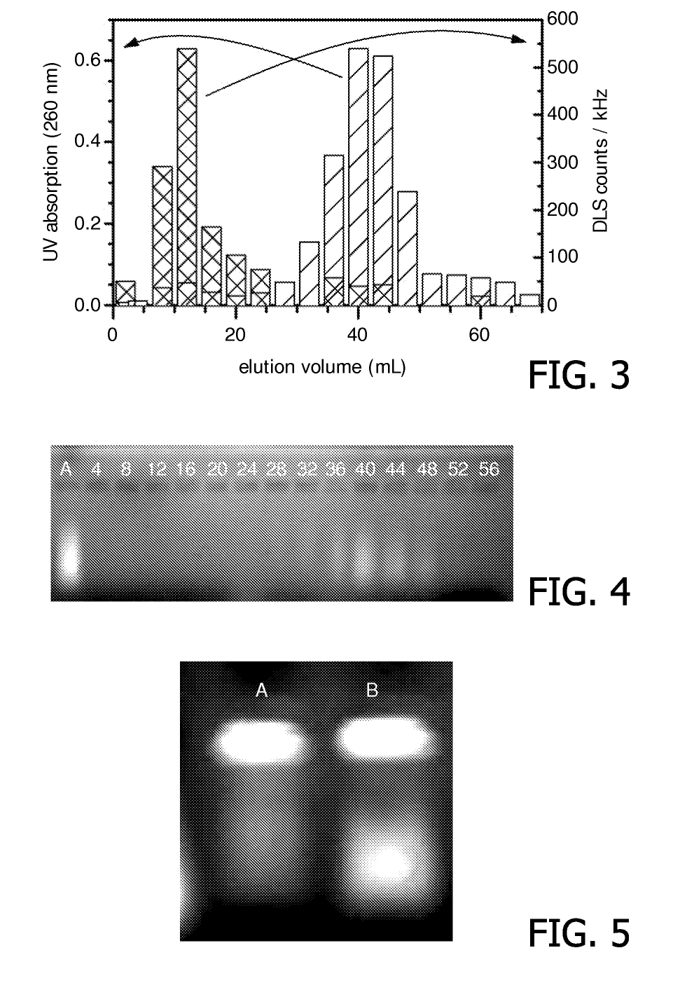

A regular glass column was loaded with Sephacryl S-1000 (GE Healthcare) to prepare for GPC (Column dimensions were: Length: 11 cm, Diameter: 3 cm; Initial Sephacryl S-1000 suspension used: 90 mL) The column was washed twice with one column volume of HEPES buffer (135 mM NaCl, 20 mM HEPES, pH 7.40). 5 mL of the crude liposome solution were carefully loaded onto the gel bed. The top of this gel bed was washed twice with 1 mL of buffer and the column was next filled with buffer. Fractions of 2 mL each were collected. The separation was controlled by dynamic light scattering (DLS) and UV-Vis spectroscopy as shown in FIG. 3. DLS is particularly suitable for the detection of liposomes, whereas UV-Vis spectroscopy is very suitable to assess the presence of DNA, since DNA has a characteristic absorption peak at 260 nm.

The successful separation of liposomal from free DNA was confirmed by agarose gel electrophoresis. A 3% agarose gel was prepared by dissolution of 1.5 g of agarose in 50 mL of buffer (0.09 M Tris-borate/0.09 M boric acid/0.001 M EDTA). The suspension was boiled in a microwave oven, until the solution became clear. The obtained solution was cooled down to about 50° C. Ethidium bromide (EB) was added to this solution in order to obtain a 0.5 μg/mL EB solution. To do so, 2.5 μL of EB (10 mg/mL) were added to the 50 mL of agarose solution. The mixture was carefully shaken to avoid the formation of air bubbles. The gel was loaded in a cassette and let set for 15 minutes. After sample loading, electrophoresis was performed at 50 V for 40 minutes. UV absorption of the gel was visualized by UV densiometry (see FIG. 4).

Under these conditions liposomes do not move on the gel, whereas free DNA moves as observed in the reference Line A loaded with a buffer solution of free herring sperm DNA. Free, unencapsulated DNA was observed for elution volumes between some 36 and 48 mL as already determined by UV analysis, DNA was, however, not observed in the earlier fractions containing liposomes. Most likely, the DNA that was encapsulated in the liposomes was not efficiently stained by EB, which is a charged, polar molecule and as such cannot diffuse easily across the lipid bilayer into the liposomes to reach those DNA molecules.

### Example 2

### Alternative Preparation of DNA-Loaded Thermosensitive Liposomes

Liposomes were prepared as in Example 1, with the difference of adding Liss Rhod PE fluorescent lipid (0.1% replacing 0.1% of DPPC) to the lipid composition in order to visualize the presence of liposomes on the agarose gel as outlined above. The chosen initial lipid concentration was 10 mM (CHCl₃), and a DNA solution containing 30 mg/mL DNA was used to form the inner water compartment. The obtained purified liposome solution was up-concentrated by a factor of 10 using 100 kDa Amicon centrifugal units.

Temperature-induced DNA delivery was tested by heating the above solution to 50° C. for 30 minutes. Gel electrophoresis was performed of solution samples before and after heating in order to investigate the efficiency of the release of entrapped DNA (see FIG. 5). Before heating (Line A) only one main spot remained at the origin of the gel and a weak background signal could be detected across the respective line. After heating (Line B), an intense additional spot was clearly detected corresponding to the released DNA. In both samples the presence of liposomes, which did not move in the gel network, was confirmed by the fluorescence labelling of the lipid double layer. After heating for 30 minutes DNA was clearly released.

### Example 3

### Verification of Drug Release

DNA/Resovist-loaded liposomes were prepared as described in Example 1 using a mixture of DNA and Resovist in the loading aqueous phase. The chosen initial lipid concentration was 10 mM (CHCl₃), and the used inner water compartment contained 15 mg/mL DNA and Resovist (0.25 mM Fe) (see Table 1). After purification, the sample was up-concentrated by a factor of 10 using 100 kDa Amicon centrifugal units.

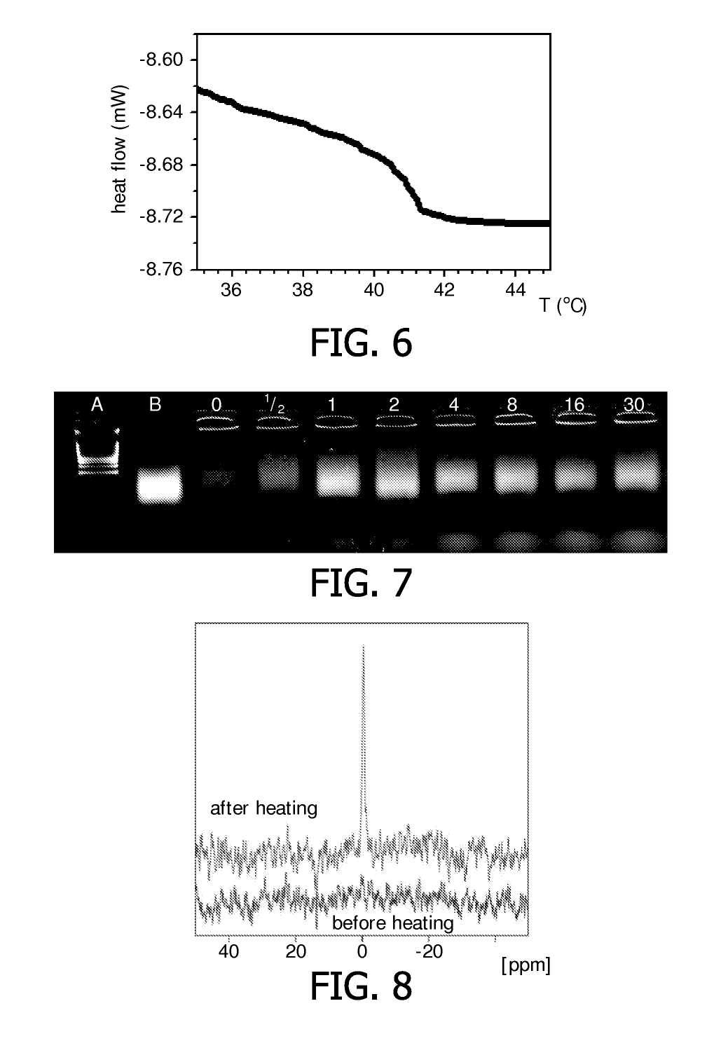

The melting phase transition temperature of the liposomal lipid bilayer was determined by means of differential scanning calorimetry (DSC). A sample was subjected to a heating/cooling cycle between 20° C. and 60° C. at a heating and cooling rate of 15° C./min and the associated heat flow was monitored. From the obtained thermogram (see FIG. 6), the melting phase transition temperature was determined to be 40.8° C. in two subsequent heating cycles, which is in good agreement with the for this lipid composition expected melting phase transition of 41° C.

Another sample of the same solution comprising DNA/Resovist-loaded liposomes was heated up at 50° C. for 30 minutes. Samples were taken at different time points between 0 and 30 min after the start of heating and subjected to gel electrophoresis. As depicted in FIG. 7, a large fraction of the entrapped DNA was already released after 30 seconds. DNA-release was essentially complete within one minute.

The release of DNA was further established by using ³¹P NMR spectroscopy (FIG. 8). The spectra of a buffered solution of DNA/Resovist-loaded liposomes were acquired before and after heating at 55° C. for 30 minutes. Before heating, no signal could be detected, probably due to extensive line broadening of the intra-liposomal DNA phosphorous atoms. The phosphorous MR resonance of DNA became visible only after heating above the melting transition temperature of the thermosensitive liposome, which therefore proves the release of DNA above the melting phase transition temperature of the thermosensitive liposomes.

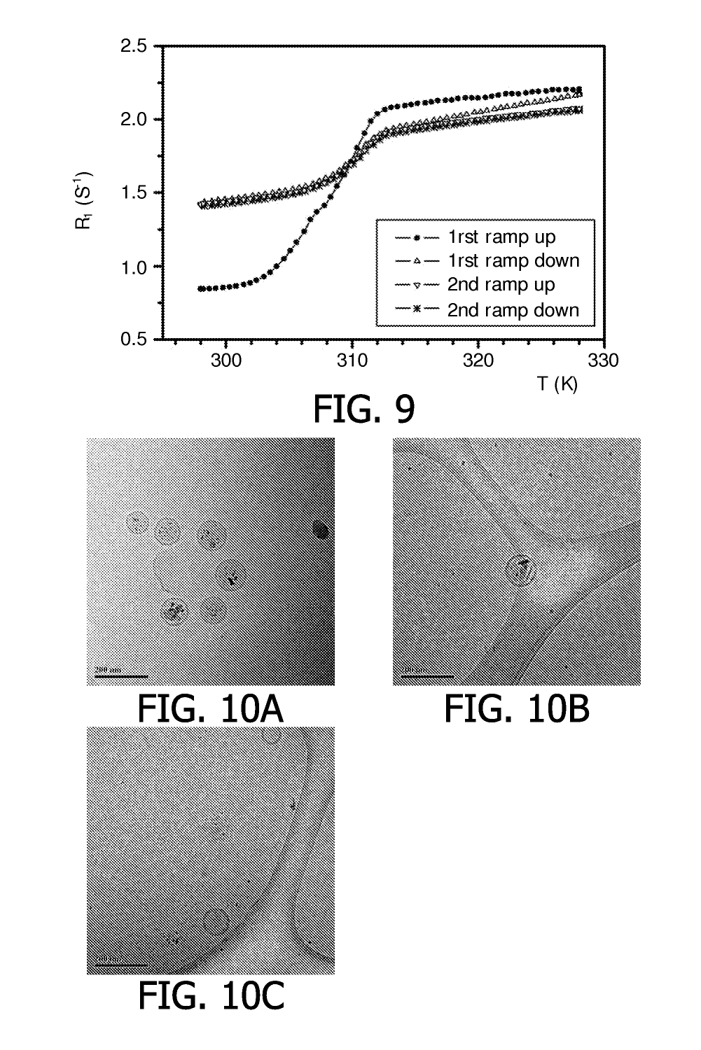

After confirming the temperature-induced release of DNA from the DNA/Resovist-loaded liposomes, in the second step the temperature-induced release of Resovist was investigated. Towards this end, the longitudinal relaxation time (T₁) of a respective solution was monitored by NMR spectroscopy as a function of temperature. Two successive heating cycles were performed (see FIG. 9) comprising the steps of, heating from RT to 55° C. with a heating rate of 0.5 K/min and subsequently cooling down to RT. It was observed that upon the first heating, a marked increase of the relaxation rate R₁ (R₁=1/T₁) was measured around the melting phase transition temperature of the lipid double layer, indicating a release of the entrapped Resovist particles. Upon cooling from 55° C. to 25° C., the R₁ decreased again around the melting phase transition temperature and reached a final value that was significantly higher (1.5 s⁻¹ vs 0.8 s⁻¹) than the initial starting value. This result shows that the release of Resovist was efficient, however, some Resovist particles may have remained inside the liposomes. A further heating cycle was carried out, indicating that the remaining R₁ change around the melting phase transition temperature is most likely due to the presence of some remaining Resovist particles encapsulated within the liposomes. The internalized liposomal water is in unhindered exchange with the bulk water only above the melting phase transition temperature, which is suitable to explain the remaining increase of the R₁ even in the second heating cycle.

As summarized in FIG. 10, the release of iron oxide particles from the liposomal carriers above their melting phase transition temperature was independently confirmed in a cryoTEM analysis. Before heating, thermosensitive liposomes with a high Resovist-load were present and no free Resovist particles were observed (A). After heating for 1 min at 50° C. (B), a fraction of the encapsulated Resovist particles was released. As a result, unencapsulated Resovist as well as both, filled and empty liposomes were observed. After 30 min of heating at the same temperature (C), only empty liposomes and free Resovist were observed, which suggests that all Resovist particles had been released. This results confirms the conclusions drawn based on the above gel analyses and NMR experiments.

In contrast to MRI, no change of the signal intensity was observed in respective magnetic particle spectroscopy (MPS, OD-MPI) experiments. The confinement of Resovist in liposomes as well as the co-confinement of DNA and Resovist in the same liposomes did not result in a change of the signal intensity when normalized to the total amount of iron (thus the total particle concentration). Consequently the release of the entrapped Resovist from thermosensitive liposomes did not cause a change of the MPS signal.

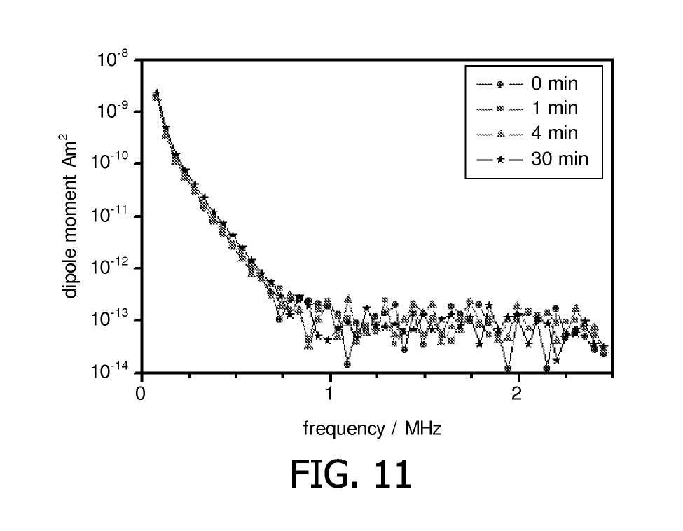

In detail, a buffered solution of liposomes loaded with DNA and Resovsist nanoparticles was heated to 50° C. Samples were collected at different time points and quickly cooled to RT in an ice bath. The measured MPS signal of the DNA/Resovist-loaded thermosensitive liposomes showed no significant changes upon heating, hence the release of entrapped Resovist from thermosensitive liposomes did not influence their MPS signal, as clearly demonstrated in FIG. 11.

1. A composition comprising a shell structure forming a cavity, wherein said shell structure comprises a drug and wherein said composition is associated with at least one contrast agent; wherein said shell structure is capable of releasing its contents into the exterior upon the application of an external stimulus and wherein said contrast agent comprises magnetic particles which are capable of being detected by Magnetic Particle Imaging (MPI), wherein at least more than 5% (w/w) of the magnetic particles comprised in said contrast agent have a magnetic moment of at least 10⁻¹⁸ m²A, wherein said magnetic particles are preferably composed of Fe, Co, Ni, Zn or Mn or alloys thereof or oxides of any of these, more preferably composed of Fe₂O₃ or Fe₃O₄.

2. The composition of claim 1, wherein at least more than 5% (w/w) of said magnetic particles have a remagnetization time of less than 10 milliseconds per particle.

3. The composition of claim 1, wherein said contrast agent is associated with the outside or with internal portions of said shell structure, is associated with said drug or is embedded within the cavity of said shell structure.

4. The composition of claim 1, wherein said shell structure constitutes a liposome, a polymersome, a nanocapsule or any mixtures thereof, preferably comprising a thermo- or pressure-sensitive material.

5. The composition of claim 1, wherein said external stimulus is capable of forming pores and/or of decomposing said shell structure.

6. The composition of claim 5, wherein said external stimulus is a temperature-increase, a temperature-decrease, a pressure-increase and/or a pressure-decrease.

7. Use of
(i) a composition comprising a shell structure forming a cavity, wherein said shell structure comprises a drug and wherein said composition is associated with at least one contrast agent, wherein said contrast agent is capable of being detected by Magnetic Particle Imaging (MPI) and wherein said shell structure is capable of releasing its contents into the exterior upon the application of an external stimulus; or
(ii) a composition as defined in claim 1,
as a carrier for a controlled delivery of a drug.

8. The use of claim 7, wherein said controlled delivery comprises a detection or localization using MPI and optionally Magnetic Resonance Imaging (MRI).

9. The use of claim 8, wherein said controlled release further comprises the release of the content(s) of said shell structure via the application of an external stimulus, preferably via a temperature-increase, a temperature-decrease, a pressure-increase and/or a pressure-decrease stimulus.

10. A method of data acquisition for the control of a drug delivery process comprising the detection or localization via MPI of
(i) a composition comprising a shell structure forming a cavity, wherein said shell structure comprises a drug and wherein said composition is associated with at least one contrast agent, wherein said contrast agent is capable of being detected by MPI and wherein said shell structure is capable of releasing its contents into the exterior upon the application of an external stimulus; or
(ii) a composition as defined in claim 1,
before, during and/or after the application of an external stimulus releasing the content(s) of said shell structure.

11. The method of claim 10, wherein said detection or localization additionally uses MRI.

12. The method of claim 10, wherein said method comprises as additional step the release of the content(s) of said shell structure via the application of an external stimulus, preferably a temperature-increase, a temperature-decrease, a pressure-increase and/or a pressure-decrease stimulus.

13. A composition comprising a shell structure forming a cavity, wherein said shell structure comprises a drug and wherein said composition is associated with at least one contrast agent, wherein said contrast agent is capable of being detected by MPI and wherein said shell structure is capable of releasing its contents into the exterior upon the application of an external stimulus; or a composition as defined in claim 1 for treating a pathological condition.

14. The composition of claim 13, wherein said drug is to be administered by the application of a stimulus, wherein said stimulus is conveyed via a local heat system, via an electrical field, via a magnetic field, via focused ultrasound radiation and/or via radiofrequency radiation, leading to the release of said drug from said shell structure into the exterior.

15. The composition of claim 13, wherein said composition is detectable or localizable with MPI and optionally MRI.
